(19) United States
(12) Patent Application Publication
(10) Pub. No.: US 2009/0148886 A1
(43) Pub. Date: Jun. 11, 2009

(54) METHODS OF IDENTIFYING COMPOUNDS THAT DECREASE INTRAOCULAR PRESSURE

(75) Inventor: Alan D. Marmorstein, Tucson, AZ (US)

Correspondence Address: KLARQUIST SPARKMAN, LLP, 121 SW SALMON STREET, SUITE 1600, PORTLAND, OR, 97204, US

(73) Assignee: The Arizona Board of Regents on behalf of the University of Arizona

(21) Appl. No.: 12/329,269

(22) Filed: Dec. 5, 2008

Related U.S. Application Data
(60) Provisional application No. 61/005,854, filed on Dec. 7, 2007.
(60) Provisional application No. 61/135,817, filed on Jul. 24, 2008.

(51) Int. Cl. C12Q 1/02

(52) U.S. Cl. 435/29

(57) ABSTRACT

This disclosure concerns methods for identifying Best2 modulators, for example methods of screening for Best2 inhibitors. In some examples, the methods include identifying compounds that alter intracellular calcium concentration, intracellular pH, or transepithelial potential in cells expressing Best2. In certain embodiments, the disclosure concerns methods of identifying compounds that decrease intraocular pressure.

### CROSS REFERENCE TO RELATED APPLICATIONS

This application claims priority to and the benefit of U.S. Provisional Application No. 61/005,854, filed Dec. 7, 2007, and U.S. Provisional Application No. 61/135,817, filed Jul. 24, 2008, both of which are incorporated herein in their entirety.

### ACKNOWLEDGMENT OF GOVERNMENT SUPPORT

This invention was made with government support under grant number R01 EY013160 awarded by the National Institutes of Health. The government has certain rights in the invention.

### FIELD

This disclosure concerns the role of Bestrophin 2 (Best2) in regulation of intraocular pressure, and in particular, methods of identifying modulators of Best2 activity that reduce intraocular pressure.

### BACKGROUND

Glaucoma is a leading cause of blindness in the United States affecting as many as 2.2 million Americans, and is the second leading cause of blindness worldwide. Approximately one half to two thirds of all glaucoma cases are associated with an increased intraocular pressure (IOP), and all current glaucoma treatment strategies aim to reduce IOP, even in patients with “normal tension” glaucoma. Typically this is accomplished by reducing the rate of aqueous flow using small molecule drugs or by enhancing drainage via drugs or surgery. While these strategies are effective in diminishing vision loss, some patients continue to lose vision despite the use of drugs to reduce IOP, and many discontinue use of these drugs because of undesirable side effects.

### SUMMARY

The inventor has surprisingly found that Bestrophin 2 (Best2) regulates intraocular pressure. For example, it is shown herein that mice lacking expression of Best2 protein have decreased intraocular pressure (IOP), which is a result of increased aqueous flow and an even greater increase in outflow facility. These results indicate that Best2 inhibitors can be therapeutically useful to decrease IOP, for example to treat glaucoma.

Disclosed herein are methods of screening to identify Best2 modulators (such as inhibitors, e.g. compounds that decrease IOP). In particular examples, the methods include contacting a cell (such as a non-pigmented epithelium cell) expressing Best2 with one or more test compounds and measuring one or more cell characteristic (for example, intracellular calcium concentration, intracellular pH, Na⁺/H⁺ exchanger (NHE) activity and/or transepithelial potential (TEP)), wherein an increase in intracellular calcium concentration or intracellular pH, or a decrease in NHE activity and/or TEP indicates that the test compound is an inhibitor of Best2 and, thus, reduces IOP. In certain embodiments, the methods can further include contacting the cell expressing Best2 with a second compound that increases intracellular calcium concentration or intracellular pH, wherein a further increase in intracellular calcium or intracellular pH in the presence of the test compound indicates that the test compound is a Best2 inhibitor.

Additional methods for identifying a test compound that inhibits Best2 are disclosed. In particular examples the methods include using an in vivo assay to determine an ocular phenotype. For example, a test compound can be contacted with a cell in a subject by administering the test compound to the subject and one or more ocular phenotypes (such as IOP, aqueous flow, outflow facility, and/or turnover of aqueous humor) can be measured, wherein a decrease in IOP or an increase in aqueous flow, outflow facility, and/or turnover of aqueous humor relative to a control identifies the test compound as one that inhibits Best2 activity.

The foregoing and other features will become more apparent from the following detailed description of several embodiments, which proceeds with reference to the accompanying figures.

### BRIEF DESCRIPTION OF THE DRAWINGS

FIG. 1A is a bar graph showing IOP of C57BL/6 mice and Best2^{+/+}, Best2^{±}, and Best2^{-/-} littermates. Box plots are shown, in which the line within the box marks the median IOP and the boundary of the box furthest from 0 indicates the 75^{th} percentile. Error bars above and below the boxes indicate the 90^{th} and 10^{th} percentiles, respectively. Data within the bracket denoted * indicate significantly reduced IOP of Best2^{-/-} mice versus the other mice.

FIG. 1B is a bar graph showing IOP of C57BL/6 mice and Best2^{+/+}, Best2^{±}, and Best2^{-/-} littermates following treatment with brinzolamide. Box plots are shown, in which the line within the box marks the median IOP and the boundary of the box furthest from 0 indicates the 75^{th} percentile. Error bars above and below the boxes indicate the 90^{th} and 10^{th} percentiles, respectively.

FIG. 1C is a bar graph showing IOP of C57BL/6 mice and Best2^{+/+}, Best2^{±}, and Best2^{-/-} littermates following treatment with timolol. Box plots are shown, in which the line within the box marks the median IOP and the boundary of the box furthest from 0 indicates the 75^{th} percentile. Error bars above and below the boxes indicate the 90^{th} and 10^{th} percentiles, respectively.

FIG. 2A is a bar graph showing rate of aqueous flow (Fₐ) in Best2^{+/+} (n=9) and Best2^{-/-} mice (n=23). Fₐ is significantly elevated (p<0.001) in Best2^{-/-} mice compared to Best2^{+/+} mice. Data are presented as a box plot in which the line within the box marks the median and the boundaries of the box indicate the range covered by the middle 50% of measurements. Bars above and below the boxes indicate the 90^{th} and 10^{th} percentiles, respectively.

FIG. 2B is a bar graph showing conventional outflow (Cₜ) in Best2^{+/+} (n=9) and Best2^{-/-} mice (n=23). Data are presented as a box plot in which the line within the box marks the median and the boundaries of the box indicate the range covered by the middle 50% of measurements. Bars above and below the boxes indicate the 90^{th} and 10^{th} percentiles, respectively. Symbols outside of the box and bars are outliers.

FIG. 3 shows photomicrographs of toluidine blue stained thick sections of the anterior chamber angle in Best2^{+/+} mice (FIG. 3A and FIG. 3C) and Best2^{-/-} mice (FIG. 3B and FIG. 3D). Schlemm's canal (sc); trabecular meshwork (tm).

FIG. 4 shows optical coherence tomography images of the anterior chamber of live Best2^{+/+} mice (FIG. 4A) and Best2^{-/-} mice (FIG. 4B). Anterior chamber depth (ACD) and corneal thickness (CT) were measured from the images.

FIG. 5A shows traces of pHᵢ in human fetal RPE (fhRPE) cells expressing endogenous Best1 (control), fhRPE cells overexpressing human Best1, and fhRPE cells overexpressing human Best1 W93C mutant following induction of an acid load with 20 mM NH₄Cl, followed by washout.

FIG. 5B is a bar graph showing resting pHᵢ in fhRPE cells expressing endogenous Best1 (control), fhRPE cells overexpressing human Best1, and fhRPE cells overexpressing human Best1 W93C mutant.

FIG. 6A shows traces of pHᵢ in fhRPE cells expressing endogenous Best1 (control), fhRPE cells overexpressing human Best1, and fhRPE cells overexpressing human Best1 W93C mutant in nominally HCO₃⁻ free HEPES buffered balanced salt solution. An acid load was induced by addition of 20 mM NH₄Cl in the absence of Na⁺.

FIG. 6B is a bar graph showing resting pHᵢ in fhRPE cells expressing endogenous Best1 (control), fhRPE cells overexpressing human Best1, and fhRPE cells overexpressing human Best1 W93C mutant in the presence or absence of HCO₃⁻. Data are mean±SD for ≧7 experiments with ≧12 cells/experiment.

FIG. 6C is a bar graph showing the effect of substitution of NMDG for Na⁺ on resting pHi in fhRPE cells expressing endogenous Best1 (control), fhRPE cells overexpressing human Best1, and fhRPE cells overexpressing human Best1 W93C mutant. Data are mean±SD for ≧7 experiments with ≧12 cells/experiment.

FIG. 6D is a graph showing net acid-base flux during recovery from an acid load in fhRPE cells expressing endogenous Best1 (control), fhRPE cells overexpressing human Best1, and fhRPE cells overexpressing human Best1 W93C mutant. Data are means for ≧7 experiments with ≧12 cells/experiment.

FIG. 7A shows representative pHᵢ recordings in eyecup preparations from wild type mice, Best1 W93C knockin (KI) mice, and Best1 knockout (KO) mice. Cells were pulsed with 20 mM NH₄Cl in the absence of Na⁺. Na⁺ was subsequently added back (arrow in KO trace) following washout of the NH₄Cl (arrowhead in KO trace).

FIG. 7B is a bar graph showing resting pHᵢ in wild type mice (n=8), Best1 W93C knockin (KI) mice (n=4 for Best1^{+/ki}; n=5 for Best1^{ki/ki}), and Best1 knockout (KO) mice (n=7). Bars indicate mean±SD.

FIG. 7C is a bar graph showing the change in pHᵢ on substitution of NMDG for Na⁺ in wild type mice (n=8), Best1 W93C knockin (KI) mice (n=4 for Best1^{+/ki}; n=5 for Best1^{ki/ki}), and Best1 knockout (KO) mice (n=7). Bars indicate mean±SD. The symbol * indicates p<0.05.

FIG. 7D is a bar graph showing Na⁺ dependent recovery from acid load in wild type mice (n=8), Best1 W93C knockin (KI) mice (n=4 for Best1^{+/ki}; n=5 for Best1^{ki/ki}), and Best1 knockout (KO) mice (n=7) at 60 seconds and 180 seconds. Bars indicate mean±SD. The symbol * indicates p<0.01 for Best1^{ki/ki}.

FIG. 8A shows average recordings of pHᵢ in HEK293 cells transfected with hBest1, mBest2, hBest1 W93C mutant (W93C), or hBest1 R218C mutant (R218C). 0 Na⁺ indicates replacement of Na⁺ in the buffer with NMDG.

FIG. 8B is a bar graph showing resting pHi in HEK293 cells transfected with hBest1 (n=4), mBest2 (n=5), hBest1 W93C mutant (W93C) (n=6), or hBest1 R218C mutant (R218C) (n=4). Bars indicate average±SD.

FIG. 8C is a bar graph showing recovery from acid load over the first 60 seconds in HEK293 cells transfected with hBest1 (n=4), mBest2 (n=5), hBest1 W93C mutant (W93C) (n=6), or hBest1 R218C mutant (R218C) (n=4). Bars indicate average±SD.

FIG. 9A is a bar graph showing transepithelial potential (TEP) of fhRPE cells expressing hBest1, hBest1 W93C mutant (W93C), or hBest1 R218C mutant (R218C).

FIG. 9B is a bar graph showing transepithelial resistance (TER) of fhRPE cells expressing hBest1, hBest1 W93C mutant (W93C), or hBest1 R218C mutant (R218C).

FIG. 9C is a bar graph showing the calculated short circuit current (I_{sc}) in of fhRPE cells expressing hBest1, hBest1 W93C mutant (W93C), or hBest1 R218C mutant (R218C).

### SEQUENCE LISTING

Any nucleic acid and amino acid sequences listed herein or in the accompanying sequence listing are shown using standard letter abbreviations for nucleotide bases, and three letter code for amino acids, as defined in 37 C.F.R. 1.822. In at least some cases, only one strand of each nucleic acid sequence is shown, but the complementary strand is understood as included by any reference to the displayed strand.

SEQ ID NOs: 1 and 2 show the nucleic acid (cDNA) and amino acid sequences, respectively, of an exemplary human Bestrophin 2.

SEQ ID NOs: 3 and 4 show the nucleic acid (cDNA) and amino acid sequences, respectively, of an exemplary mouse Bestrophin 2.

### DETAILED DESCRIPTION

### I. Abbreviations and Terms

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety for all purposes. In case of conflict, the present specification, including explanations of terms, will control. In addition, the materials, methods, and examples are illustrative only and not intended to be limiting.

Definitions of common terms in molecular biology may be found in Benjamin Lewin, Genes V, published by Oxford University Press, 1994 (ISBN 0-19-854287-9); Kendrew et a. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

ACD: anterior chamber depth

Best2: bestrophin 2 gene or protein

C: outflow facility

EVP: episcleral venous pressure

Fₐ: aqueous flow

F_{c}: conventional outflow

Fᵤ: uveoscleral outflow

IOP: intraocular pressure

I_{sc}: short circuit current

NHE: Na⁺/H⁺ exchanger

NMDG: N-methyl-D-glucamine

NPE: non-pigmented epithelium

pHᵢ: intracellular pH

RPE: retina pigmented epithelium

SNARF-1: seminaphtorhodafluor-1

TEP: transepithelial potential

TER: transepithelial resistance

Vₐ: aqueous volume

Aqueous humor: The fluid in the anterior chamber of the eye. Aqueous humor provides nutrients to the lens and corneal endothelium, and its pressure maintains the convex shape of the cornea. It is produced by the combined effect of the pigmented and non-pigmented epithelium of the ciliary body. Aqueous humor is continually produced by the ciliary processes, and this rate of production must be balanced by a substantially equal rate of aqueous humor drainage. Small variations in the changes in production or outflow of aqueous humor may have a large influence on the intraocular pressure. The dynamic amount of aqueous humor that has been produced less that resorbed by the ciliary body is the aqueous flow (Fₐ).

The drainage route for aqueous humor flow is first through the posterior chamber, then the narrow space between the posterior iris and the anterior lens (contributes to small resistance), then through the pupil to enter the anterior chamber. From there, the aqueous humor exits the eye through the trabecular meshwork into Schlemm's canal, then flows through 25-30 collector canals into the episcleral veins. The greatest resistance to aqueous flow is provided by the trabecular meshwork, and this is where most of the aqueous outflow occurs. A secondary route is the uveoscleral drainage, which is independent of the intraocular pressure.

Bestrophin 2 (Best2): An approximately 57 kDa protein that is expressed predominantly in the eye and colon (Stohr et al., Eur. J. Hum. Genet. 10:281-284, 2002). In the eye, Best2 is specifically expressed in the non-pigmented epithelium (NPE) of the ciliary body. Best2 is also expressed in colon epithelium, salivary gland acinar cells, and olfactory epithelium.

Best2 sequences are publicly available. For example, GenBank Accession number NC_{—}000019.8 (12724407 . . . 12730272) (Mar. 3, 2008) discloses an exemplary human Best2 gene sequence (incorporated herein by reference). GenBank Accession numbers NM_{—}017682.2 (May 1, 2008) and NP_{—}060152.2 (May 1, 2008) disclose exemplary human Best2 cDNA and protein sequences, respectively (both incorporated herein by reference; SEQ ID NOs: 1 and 2, respectively). GenBank Accession number NC_{—}000074.5 (87531101 . . . 87537490) (Jul. 10, 2007) discloses an exemplary mouse Best2 gene sequence (incorporated herein by reference). GenBank Accession numbers NM_{—}145388.3 (Jul. 20, 2008) and NP_{—}663363.2 (Jul. 20, 2008) disclose exemplary mouse Best2 cDNA and protein sequences, respectively (both incorporated herein by reference; SEQ ID NOs: 3 and 4, respectively). One skilled in the art will appreciate that Best2 nucleic acid and protein molecules can vary from those publicly available, such as Best2 sequences having one or more substitutions (for example conservative substitutions), deletions, insertions, or combinations thereof, while still retaining Best2 biological activity. In addition, Best2 molecules include fragments that retain the desired Best2 biological activity.

Cell characteristic: An aspect or characteristic of a cell, such as a parameter of cell physiology. The cell characteristic is a parameter that is directly or indirectly under the influence of Bestrophin 2. Exemplary cellular parameters include, but are not limited to intracellular calcium concentration, intracellular pH, NHE activity, and electrophysiological characteristics (for example transepithelial potential, transepithelial resistance and/or I_{sc}). Determining a cell characteristic is accomplished by measuring the parameter of interest by any method known to one of skill in the art. In some examples, a test compound that alters one or more cell characteristic (such as increasing or decreasing intracellular calcium, intracellular pH, NHE activity, TEP, or other cell characteristic) is a compound that modulates Best2 activity. In particular examples, a compound that increases a cell characteristic (such as intracellular calcium or intracellular pH) is a compound that inhibits Best2. In other examples, a compound that decreases a cell characteristic (such as NHE activity or TEP) is a compound that inhibits Best2.

Conservative substitution: A substitution of an amino acid residue for another amino acid residue having similar biochemical properties. Typically, conservative substitutions have little to no impact on the biological activity of a resulting polypeptide. In a particular example, a conservative substitution is an amino acid substitution in a peptide that does not substantially affect the biological function of the peptide. A peptide can include one or more amino acid substitutions, for example 2-10 conservative substitutions, 2-5 conservative substitutions, 4-9 conservative substitutions, such as 2 or 5. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain: (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which: (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Contacting: Placement in direct physical association, which can include both in solid and liquid form. Contacting can occur in vitro for example with samples, such as biological samples, for example cells or cell free extracts, such as cell lysates, or in vivo by administering to a subject. In some examples, a sample (such as a cell expressing Best2) is contacted with a test compound, such as a test compound that modulates Best2 activity.

Glaucoma: A group of diseases of the optic nerve characterized by progressive loss of optic nerve axons and visual field damage. Glaucoma is the second leading cause of blindness world wide. The major risk factor for most glaucoma is increased IOP, although glaucoma that is not accompanied by an increase in IOP (normal tension glaucoma) also occurs. There are two main types of glaucoma, open angle glaucoma and closed angle glaucoma. Open angle glaucoma is caused by blockage of the outflow pathway(s), which is where the aqueous humor in the eye drains out. Because the outflow of aqueous humor is blocked, the pressure builds up in the eye and causes damage to the optic nerve. However, approximately one-third of open angle glaucoma is not associated with an increase in IOP. Closed angle glaucoma is caused by contact between the iris and trabecular meshwork, which in turn obstructs outflow of the aqueous humor from the eye. In over half of all cases, prolonged contact between iris and TM causes the formation of synechiae, causing permanent obstruction of aqueous outflow.

Current treatments for glaucoma focus on the reduction of IOP to slow or prevent damage to the optic nerve. Medications used to treat glaucoma include prostaglandin analogs (such as latanoprost, bimatoprost, or travoprost), topical beta-adrenergic receptor antagonists (such as timolol, levobunolol, or betaxolol), alpha2-adrenergic agonists (such as bimonidine), sympathomimetics (such as epinephrine or dipivefrin), miotic agents (such as pilocarpine), and carbonic anhydrase inhibitors (such as dorzolamide, brinzolamide, and acetazolamide). Surgical intervention may also be used to reduce IOP, including trabeculectomy, canaloplasty, laser trabeculoplasty, and laser peripheral iridotomy.

Intracellular calcium: Concentration of calcium (such as Ca²⁺) in a cell, such as in the cytosol or other cellular compartment. Methods of determining intracellular calcium concentration are well known to one of skill in the art. In some examples, a cell of interest (such as a cell expressing Best2) is loaded with a calcium-sensitive dye (such as a fluorescent or luminescent compound). The cell is treated, for example, with a test compound and fluorescence or luminescence is measured. In some examples, a compound that increases intracellular calcium concentration is an inhibitor of Best2.

Intracellular pH (pHᵢ): pH of intracellular fluid, for example, the cytosol. Methods of determining pHᵢ are well known to one of skill in the art. In some examples, a cell of interest (such as a cell expressing Best2) is loaded with a pH-sensitive dye (such as a fluorescent or luminescent compound). The cell is treated, for example, with a test compound and fluorescence or luminescence is measured. In some examples, a compound that increases pHᵢ is an inhibitor of Best2.

Intraocular pressure (IOP): A measurement of fluid pressure inside the eye. IOP is a function of production of liquid aqueous humor by the ciliary body of the eye and its drainage through the trabecular meshwork. Aqueous humor flows from the ciliary bodies into the posterior chamber, which is bounded posteriorly by the lens and the zonule of Zinn and anteriorly by the iris. It then flows through the pupil of the iris into the anterior chamber, which is bounded posteriorly by the iris and anteriorly by the cornea. From there the trabecular meshwork drains aqueous humor via Schlemm's canal into scleral plexuses and general blood circulation.

IOP is commonly measured in human subjects by tonometry (for example, Goldmann tonometry or non-contact tonometry). In animal subjects, IOP can also be measured by a method utilizing cannulation of the anterior chamber of the eye (see, e.g., Aihara et al., Invest. Ophthalmol. Vis. Sci. 44:5168-5173, 2003).

Non-pigmented epithelium (NPE): The inner layer of the ciliary body epithelium. The outer layer is pigmented, while the inner layer is unpigmented. The NPE is involved in generation of aqueous humor. Best2 is expressed in the ciliary body, particularly the basolateral plasma membrane of the NPE.

Ocular phenotype: A characteristic or trait of the eye. The ocular phenotype is a characteristic that is directly or indirectly under the influence of Bestrophin 2, such as a characteristic related to the production, drainage, or turnover of the aqueous humor. In some examples, an ocular phenotype includes intraocular pressure, aqueous flow (such as rate of aqueous humor formation), outflow facility (such as total outflow volume, conventional outflow and uveoscleral outflow), and turnover rate of aqueous humor.

Outflow facility: The drainage pathway of aqueous humor from the anterior chamber of the eye. There are two pathways of the outflow facility, the conventional outflow and the uveoscleral outflow. The conventional outflow is pressure-dependent and provides a majority of the aqueous drainage through the trabecular meshwork in humans. In mice, the conventional outflow accounts for only about 20% of the aqueous drainage. The uveoscleral outflow is pressure-independent and provides the remaining aqueous outflow through the ciliary body face and iris.

Peptide, Polypeptide, and/or Protein: Any compound composed of amino acids, amino acid analogs, chemically bound together. Amino acids generally are chemically bound together via amide linkages (CONH). Additionally, amino acids may be bound together by other chemical bonds. For example, the amino acids may be bound by amine linkages. Peptides include oligomers of amino acids, amino acid analog, or small and large peptides, including polypeptides or proteins. In some examples, a peptide is a Best2 peptide.

Sample: A specimen that includes biological materials, such as isolated proteins. Examples include, but are not limited to, cells or fractions thereof, such as a cell expressing Best2 or a cell lysate of a cell expressing Best2. In one example, a sample is a cell-free sample, such as one that includes isolated or purified proteins (such as Best2). In a specific example, a sample consists essentially of purified proteins in solution (e.g., a buffer solution appropriate for incubating proteins in).

Subject: Living multi-cellular vertebrate organisms, a category that includes both human and non-human mammals. The methods disclosed herein have equal applications in medical and veterinary settings. Therefore, the general term “subject” is understood to include all animals, including, but not limited to, humans or veterinary subjects, such as other primates, mice, rats, dogs, cats, pigs, horses, and cows.

Test compound: Any substance or any combination of substances that is useful for achieving an end or result. The compounds identified using the methods disclosed herein can be of use for affecting the activity of Best2, such as the ability of Best2 to regulate intracellular calcium, intracellular pH, intraocular pressure, or aqueous flow, and can be of use for treating glaucoma. Any compound that has potential (whether or not ultimately realized) to affect the activity of Best2, including, but not limited to the regulation of intracellular calcium or pH, can be tested using the methods of this disclosure.

Transepithelial potential (TEP): Difference in potential across an epithelium (such as a ciliary body epithelial bilayer) due to differences in the membrane potential of the apical and basolateral plasma membranes. In some examples, the TEP of an isolated ciliary epithelial bilayer is approximately −650 μV (see, e.g., Sears et al., Tr. Am. Ophth. Soc. LXXXIX:131-154, 1991).

### II. Overview of Several Embodiments

Disclosed herein are methods of screening to identify Best2 modulators (e.g., for decreasing IOP). These methods can include contacting (e.g., incubating or treating) a cell (such as a NPE cell) expressing Best2 with one or more test compounds, determining whether the test compound alters one or more cell characteristic, and selecting a compound that alters the cell characteristic in the presence of the compound as compared to in the absence of the compound. In some examples, determining whether the test compound alters a cell characteristic includes measuring one or more of intracellular calcium concentration, intracellular pH, NHE activity, and TEP, wherein a change (such as an increase) in intracellular calcium concentration and/or intracellular pH, or a change (such as a decrease) in NHE activity and/or TEP in the presence of the test compound relative to in the absence of the test compound indicates that the test compound is an inhibitor of Best2 and thus, a compound that decreases IOP.

In additional examples, the cell expressing Best2 is further contacted with a second compound (for example, before or concurrent with contacting the test compound with the cell). The second compound is a compound that increases intracellular calcium (such as ATP, dopamine, or epinephrine) or a compound that increases intracellular pH (such as NH₄Cl). The cell characteristic is then measured, and a compound that changes (such as increases) intracellular calcium concentration and/or intracellular pH in the presence of the test compound and the second compound as compared to in the absence of the test compound indicates that the compound is an inhibitor of Best2, and thus a compound that decreases IOP.

Certain embodiments of the methods also include selecting a test compound indicated to be a Best2 modulator (such as a Best2 inhibitor) for further analysis. For example, if a test compound is indicated to be an Best2 inhibitor in vitro, such agents can be further analyzed in vivo (e.g., in an animal model).

In particular examples, the cell is in a subject, such as a laboratory mammal (for example, a mouse, rat, rabbit, or other animal model) or a human subject, and contacting the cell with the test compound includes administering the test compound to the subject. For example, a test compound can be administered to a subject and one or more ocular phenotypes (such as IOP, aqueous humor production or flow, outflow facility, or turnover of aqueous humor) can be determined and a compound that alters the ocular phenotype is selected, wherein a compound that decreases IOP, increases aqueous production, increases outflow facility and/or increases turnover of aqueous humor in the presence of the test compound as compared to in the absence of the test compound indicates that the compound is an inhibitor of Best2.

In some examples, a combination of in vitro and in vivo assays may be used, for example, determining the effect of a test compound on one or more cell characteristics, followed by determining the effect of a test compound on one or more ocular phenotypes.

In certain examples of the method, the test compound is a Best2 inhibitor. In particular examples, the test agent is a small molecule inhibitor of Best2 activity, an antibody that blocks Best2 activity, or a siRNA that lowers Best2 expression levels in cells.

### III. Exemplary Assays

Several methods are available for measuring Best2 biological activity. Although specific examples are provided below and elsewhere in the application, the methods are not limited to specific methods of measuring Best2 biological activity. In some examples, determining whether the test compound modulates Best2 activity in a cell includes determining a control level of Best2 activity in the cell before contacting (e.g., incubating or treating) the cell with the test compound, contacting the cell with the test compound, and determining whether contacting the cell with the test compound alters (e.g., increases or decreases) Best2 activity in the cell as compared to the control level of Best2 activity. In some examples, decreased Best2 activity in the cell in the presence of the test compounds (such as a decrease of at least 20%, at least 40%, at least 50%, at least 80%, at least 90% or even at least 99%) relative to the control level indicates that the test compound is an inhibitor of Best2. In other examples, increased Best2 activity in the cell in the presence of the test compounds (such as an increase of at least 20%, at least 40%, at least 50%, at least 80%, at least 90% or even at least 99%) relative to the control level indicates that the test compound is an activator of Best2. In some examples, Best2 activity is determined by measuring a cell characteristic that is directly or indirectly under the influence of Best2 (such as intracellular calcium concentration, intracellular pH, NHE activity, or TEP) or an ocular phenotype that is directly or indirectly under the influence of Best2 activity (such as IOP, aqueous humor production or flow, outflow facility, or turnover of aqueous humor).

A. In vitro Assays

In some examples, Best2 modulators (such as compounds that decrease intraocular pressure) are identified by measuring intracellular calcium concentration. Methods of measuring intracellular calcium concentration are routine, and the disclosure is not limited to particular methods. Cells expressing Best2 are contacted with a test compound and intracellular calcium concentration is measured. For example, cells can be incubated with varying concentrations of the test compound(s) (such as a serial dilution from 100 mM to 1 nM) for at least 10 minutes (such as at least 30 minutes, at least 1 hour, at least 4 hours, at least 24 hours, or at least 48 hours) at 37° C. Intracellular calcium concentration is then measured. In one example, measuring intracellular calcium concentration includes determining whether contacting the cell with the test compound increases intracellular calcium concentration (such as an increase of at least about 20%, at least about 50%, at least about 100%, at least about 200%, at least about 300%, at least about 400%, or at least about 500%) as compared to intracellular calcium concentration in the absence of the test compound.

In additional examples, cells expressing Best2 are contacted with a compound that increases intracellular calcium concentration (such as adenosine triphosphate, dopamine, or epinephrine). During, or at a subsequent time, the cells are incubated in the presence or absence of the test compound for a period sufficient for the intracellular calcium concentration to increase. For example, cells can be incubated with varying concentrations of the test compound(s) (such as a serial dilution from 100 mM to 1 nM) for at least 10 minutes (such as at least 30 minutes, at least 1 hour, at least 4 hours, at least 24 hours, or at least 48 hours) at 37° C. Intracellular calcium concentration is then measured. In one example, measuring intracellular calcium concentration includes determining whether contacting the cell with the test compound increases intracellular calcium concentration (such as an increase of at least about 20%, at least about 50%, at least about 100%, at least about 200%, at least about 300%, at least about 400%, or at least about 500%) as compared to intracellular calcium concentration in the absence of the test compound.

Any method known to one of ordinary skill in the art can be used to measure intracellular calcium concentration. In some examples cells expressing Best2 (such as NPE cells) are loaded with a calcium-sensitive dye (such as a dye that exhibits increased fluorescence or luminescence upon binding calcium). The cells are contacted with a test compound (in the presence or absence of a compound that increases intracellular calcium) and fluorescence or luminescence is measured (such as with a fluorometer or luminometer). Examples of fluorescent calcium-sensitive dyes include, but are not limited to, fura-2, calcein, fluo-3, indo-1, and quin-2. Examples of luminescent calcium-sensitive dyes include, but are not limited to, coelenterazine and coelenterazine derivatives.

In some examples, intracellular calcium concentration can be measured by assays described herein in the same sample or a different sample that has been contacted with the given test compound. A test compound that results in an increase of at least about 20% (such as at least about 50%, at least about 100%, at least about 200%, at least about 300%, at least about 400%, or at least about 500%) in intracellular calcium concentration relative to a control or reference value (or range of values) indicates that the test compound can inhibit Best2 activity. Exemplary controls/reference values include the expected response in the absence of the test compound (e.g., an amount or range of amounts of intracellular calcium concentration expected in the absence of treatment with the test compound).

In some examples, Best2 modulators (such as compounds that decrease intraocular pressure) are identified by measuring intracellular pH. Methods of measuring intracellular pH are routine, and the disclosure is not limited to particular methods. Cells expressing Best2 are contacted with a test compound and intracellular pH is measured. For example, cells can be incubated with varying concentrations of the test compound(s) (such as a serial dilution from 100 mM to 1 nM) for at least 10 minutes (such as at least 30 minutes, at least 1 hour, at least 4 hours, at least 24 hours, or at least 48 hours) at 37° C. Intracellular pH is then measured. In one example, measuring intracellular pH includes determining whether contacting the cell with the test compound changes (such as increases) intracellular pH (for example, pHᵢ is more basic) as compared to intracellular pH in the absence of the test compound.

In additional examples, cells expressing Best2 are contacted with a compound (such as ammonium chloride) that increases intracellular pH. During, or at a subsequent time, the cells are incubated in the presence or absence of the test compound for a period sufficient for the intracellular pH to increase. For example, cells can be incubated with varying concentrations of the test compound(s) (such as a serial dilution from 100 mM to 1 nM) for at least 10 minutes (such as at least 30 minutes, at least 1 hour, at least 4 hours, at least 24 hours, or at least 48 hours) at 37° C. Intracellular pH is then measured. In one example, measuring intracellular pH includes determining whether contacting the cell with the test compound increases intracellular pH (becomes more basic) as compared to intracellular pH in the absence of the test compound.

Any method known to one of ordinary skill in the art can be used to measure intracellular pH. In some examples cells expressing Best2 (such as NPE cells) are loaded with a pH indicator dye (such as a dye that exhibits pH-sensitive fluorescence). The cells are contacted with a test compound (in the presence or absence of a compound that increases intracellular pH) and fluorescence is measured (such as using a fluorometer, microscopy, or flow cytometry). Examples of fluorescent pH indicator dyes include, but are not limited to, seminaphtorhodafluor-1 (SNARF-1), 2′,7′-bis(2-carboxyethyl)-5(6)-carboxyfluorescein (BCECF), and 5-chloromethylfluorescein diacetate (CMFDA).

In some examples, intracellular pH can be measured by assays described herein in the same sample or a different sample that has been contacted with the given test compound. A test compound that results in at least a 1% increase (such as an at least 2%, at least 3%, at least 4%, at least 5%, at least 10%, or at least 20% increase) in intracellular pH relative to a control or reference value (or range of values) indicates that the test compound can inhibit Best2 activity. In other examples, a test compound that results in at least a 1% decrease (such as an at least 2%, at least 3%, at least 4%, at least 5%, at least 10%, or at least 20% decrease) in intracellular pH relative to a control or reference value (or range of values) indicates that the test compound can inhibit Best2 activity. Exemplary controls/reference values include the expected intracellular pH in the absence of the test compound (e.g., an amount or range of amounts of intracellular pH expected in the absence of treatment with the test compound).

In some examples, Best2 modulators (such as compounds that decrease intraocular pressure) are identified by measuring Na⁺/H⁺ exchanger (NHE) activity. Methods of measuring NHE activity are routine, and the disclosure is not limited to particular methods. Cells expressing Best2 are contacted with a test compound and NHE activity is measured. For example, cells can be incubated with varying concentrations of the test compound(s) (such as a serial dilution from 100 mM to 1 nM) for at least 10 minutes (such as at least 30 minutes, at least 1 hour, at least 4 hours, at least 24 hours, or at least 48 hours) at 37° C. NHE activity is then measured. In one example, measuring NHE activity includes determining whether contacting the cell with the test compound decreases NHE activity as compared to NHE activity in the absence of the test compound.

Any method known to one of ordinary skill in the art can be used to measure NHE activity. NHE activity may be measured as the rate of Na⁺-dependent recovery of pHᵢ following an acid load (see, e.g., Di Sole et al., J. Biol. Chem. 279:2962-2974, 2004). In some examples, cells expressing Best2 are acid-loaded by treatment with NH₄Cl followed by washout. The cells are treated with a test compound and recovery from the acid load is measured. In some cases Na⁺ is substituted with an ion such as N-methyl-D-glucamine (NMDG). NHE activity is then defined as occurring following acid loading and restoration of Na⁺ to the medium. In some examples, NHE activity is measured in the presence or absence of HCO₃.

In some examples, NHE activity can be measured by assays described herein in the same sample or a different sample that has been contacted with the given test compound. A test compound that results in at least a 10% decrease (such as an at least 20%, at least 30%, at least 50%, at least 75%, at least 90%, at least 95%, or even at least 99% decrease) in NHE activity relative to a control or reference value (or range of values) indicates that the test compound can inhibit Best2 activity. Exemplary controls/reference values include the expected NHE activity in the absence of the test compound (e.g., an amount or range of amounts of NHE activity expected in the absence of treatment with the test compound).

In additional examples, Best2 modulators (such as compounds that decrease intraocular pressure) are identified by measuring transepithelial potential (TEP) of a cell monolayer, such as an NPE monolayer. Methods of measuring TEP are routine, and the disclosure is not limited to particular methods. Cells expressing Best2 are contacted with a test compound and TEP is measured. For example, cells can be incubated with varying concentrations of the test compound(s) (such as a serial dilution from 100 mM to 1 nM) for at least 10 minutes (such as at least 30 minutes, at least 1 hour, at least 4 hours, at least 24 hours, or at least 48 hours) at 37° C. TEP is then measured. In one example, measuring TEP includes determining whether contacting the cell with the test compound decreases the TEP as compared to the TEP in the absence of the test compound.

Any method known to one of ordinary skill in the art can be used to measure TEP. In some examples cells expressing Best2 (such as HT-29 cells or cells transfected with Best2) are cultured on Millicell® inserts and mounted in a water jacketed Ussing chamber (see, e.g., Oakley et al., Invest. Ophthalmol. Vis. Sci. 16:771-774, 1977; Miller and Steinberg, J. Membr. Biol. 36:337-372, 1977). TEP may be recorded using Ag/AgCl pellet electrodes bridged with agar 0.5 M KCl bridges in each bath. In addition, the transepithelial resistance (TER) of the cells may be determined by passing current (such as bipolar 10 μA pulses) across the monolayer every 30 seconds between Ag—AgCl pellets placed in each bath and measuring the current-induced voltage changes across the monolayer. I_{sc} may be calculated as I_{sc}=(TEP/TER), or can alternately be determined from the current required to clamp an epithelium at a TEP of 0 mV. The cells are contacted with a test compound and TEP, TER, and/or ISC are measured.

In some examples, TEP can be measured by assays described herein in the same sample or a different sample that has been contacted with the given test compound. A test compound that results in at least a 10% decrease (such as an at least 20%, at least 30%, at least 40%, at least 50%, at least 60%, at least 70%, at least 80%, at least 90%, or at least 95% decrease) in TEP relative to a control or reference value (or range of values) indicates that the test compound can inhibit Best2 activity. Exemplary controls/reference values include the expected TEP in the absence of the test compound (e.g., an amount or range of amounts of TEP expected in the absence of treatment with the test compound).

B. In vivo Assays

Inhibiting Best2 to increase intracellular calcium concentration or pH has advantageous effects as described herein. Thus, it may be beneficial, in some instances, to further determine whether the effect(s) of a compound identified in some described method embodiments decreases intraocular pressure in vivo. Thus, it may be beneficial (although optional) to further screen compounds identified in some method embodiments for their potential to treat or prevent glaucoma (for example, by decreasing IOP), in a subject; for example, by administering a candidate compound to a subject (such as an animal model, such as a mouse, rat, rabbit, pig, or non-human primate model, or a human subject) and determining whether IOP is decreased by the candidate compound. Animal models of glaucoma are known to one of skill in the art. Exemplary animal models include mouse models (for example, DBA/2J mice), rat models (see, e.g. Morrison, et al., Exp. Eye Res. 64:85-96, 1997; Levkovitch-Verbin et al., Invest. Ophthalmol. Vis. Sci. 43:402-410, 2002; Ueda et al., Jpn. J. Ophthalmol. 42:337-344, 1998; Yu et al., Exp. Eye Res. 83:758-770, 2006), canine models (such as primary open angle glaucoma beagles; Samuelson et al., Invest. Ophthalmol. Vis. Sci. 30:550-561, 1989), rabbit models (see, e.g., Shah et al., Ind. J. Pharmacol. 31:110-115, 1999; ), and non-human primate models (see, e.g., Hare et al., Invest. Ophthalmol. Vis. Sci. 45:2625-2639, 2004). A candidate compound that decreases IOP may be considered as a compound having potential to treat glaucoma.

The test compound may be administered to the subject by any route. In particular examples, the compound is administered topically, such as topical ocular administration, for example in the form of eye drops. In additional examples, the compound is administered intraocularly, for example by injection to the retina or ciliary body. Appropriate routes and dosages of administration can be determined by one of skill in the art.

In some examples, IOP is evaluated following treatment with a test compound. Methods of determining IOP are well known in the art. In subjects, such as laboratory mammals (for example, mice, rats, or rabbits), IOP may be evaluated by cannulating the anterior chamber with a microneedle connected to a pressure transducer (see e.g., John et al., Invest. Ophthalmol. Vis. Sci. 38:249-253, 1997; Aihara et al., Invest. Ophthalmol. Vis. Sci. 43:146-150, 2002; Aihara et al., Invest. Ophthalmol. Vis. Sci. 44:5168-5173, 2003).

In subjects, such as human or laboratory mammal subjects, IOP may be evaluated by tonometry. In some examples, IOP is measured by applanation tonometry (such as Goldmann tonometry) or non-contact tonometry. Applanation tonometry measures approximate IOP by the force required to flatten a constant area of the cornea. Non-contact tonometry (air-puff tonometry) uses a rapid air pulse to applanate the cornea. Corneal applanation is detected via an electro-optical system. IOP is estimated by detecting the force of the air jet at the instance of applanation. Current non-contact tonometers have been shown to correlate well with Goldmann tonomtery measurements. Additional tonometry methods are well known to one of skill in the art, including dynamic contour tonometry, transpalpebral tonometry, impression tonometry, pneumatonometry, and rebound tonometry.

In some examples, IOP can be measured by assays described herein in the same subject or a different subject that has been contacted with the given test compound A test compound that results in at least a 5% decrease (such as an at least 5%, at least 6%, at least 7%, at least 8%, at least 9%, at least 10%, at least 15%, or at least 20% decrease) in IOP relative to a control subject which has not been contacted with the test compound or relative to the same subject prior to administration of the test compound indicates that the test compound can decrease IOP. Exemplary controls/reference values include the expected IOP in the absence of the test compound (e.g., an amount or range of amounts of IOP expected in the absence of treatment with the test compound).

In additional examples, aqueous flow is evaluated following treatment with a test compound. Methods of determining aqueous flow are well known in the art. For example, the rate of aqueous humor formation may be determined by dilution of a fluorescent perfusate, such as a perfusate containing fluorescein isothiocyanate (FITC)-dextran or rhodamine-dextran (see e.g., Aihara et al., Invest. Ophthalmol. Vis. Sci. 44:5168-5173, 2003). Aqueous flow (Fₐ) may then be calculated based on the ratio of the fluorescent compound in the perfusion outflow (Cₒ) to the concentration of the fluorescent compound in the perfusion inflow fluid (Cᵢ) according to the formula:

Fₐ=3 μl min⁻¹ (1−CₒCᵢ).

In other examples, aqueous flow can be measured utilizing fluorophotometry (see, e.g., U.S. Pat. No. 4,573,778; Larsson et al., Arch. Ophthalmol. 116:19-24, 1998). Briefly, eyedrops containing fluorescein (such as 2-10% fluorescein) are administered to a subject. Fluorescence is measured at repeated time intervals (such as every hour or every other hour) for a period of time (such as about six to twelve hours) with a fluorophotometer. Aqueous flow is calculated from the clearance of fluorescein with the equation:

Flow=ΔM/(C_{a×Δt})−0.25 μl/min

where ΔM is the loss of mass of fluorescein in the combined cornea and anterior chamber in an interval and Cₐ is the average concentration of fluorescein during an interval. The presumed rate of diffusional clearance (0.25 μl/min) is subtracted.

In some examples, aqueous flow can be measured by assays described herein in the same subject or a different subject that has been contacted with the given test compound A test compound that results in at least a 10% increase (such as an at least 25%, at least 50%, at least 75%, at least 100%, or at least 200% increase) in aqueous flow relative to a control subject which has not been contacted with the test compound or relative to the same subject prior to administration of the test compound indicates that the test compound is an inhibitor of Best2 and thus is a compound that decreases IOP. Exemplary controls/reference values include the expected aqueous flow in the absence of the test compound (e.g., an amount or range of amounts of aqueous flow expected in the absence of treatment with the test compound).

In additional examples, outflow facility is evaluated following treatment with a test compound. Methods of determining outflow facility (C) are well known in the art. For example, using the infusion system used to measure IOP and aqueous production (see e.g., Aihara et al., Invest. Ophthalmol. Vis. Sci. 44:5168-5173, 2003), all fluid is replaced with physiological saline, and total outflow volume (Vₜ) is measured for a period of 10 minutes at two different levels of IOP (such as 25 mm Hg and 35 mm Hg). Outflow facility (C) is calculated by the following formula:

C=0.01×(Vₜ₌₂₅−Vₜ₌₃₅) μl min⁻¹/mm Hg.

In other examples, outflow facility can be determined by fluorophotometry and/or tonography (see, e.g., Hayashi et al., Exp. Eye Res. 48:621-625, 1989; Toris et al., Arch. Ophthalmol. 122:1782-1787, 2004).

In some examples, outflow facility can be measured by assays described herein in the same subject or a different subject that has been contacted with the given test compound A test compound that results in at least a 10% increase (such as an at least 25%, at least 50%, at least 75%, at least 100%, or at least 200% increase) in outflow facility relative to a control subject which has not been contacted with the test compound or relative to the same subject prior to administration of the test compound indicates that the test compound is an inhibitor of Best2 and thus is a compound that decreases IOP. Exemplary controls/reference values include the expected outflow facility in the absence of the test compound (e.g., an amount or range of amounts of outflow facility expected in the absence of treatment with the test compound).

In particular examples, a compound may be identified as a compound that decreases IOP by determining the effect of a test compound on both aqueous flow and outflow facility. A compound that increases aqueous flow and also increases outflow facility to a greater extent than the increase in aqueous flow is a compound that decreases IOP. Such an increase is reflected in an increase in the aqueous humor turnover rate, which can be calculated with the following formula:

Turnover rate (%/min)=100×Fₐ/Vₐ.

In some examples, aqueous humor turnover rate can be measured by assays described herein in the same subject or a different subject that has been contacted with the given test compound A test compound that results in at least a 10% increase (such as an at least 25%, at least 50%, at least 75%, at least 100%, or at least 200% increase) in aqueous humor turnover relative to a control subject which has not been contacted with the test compound or relative to the same subject prior to administration of the test compound indicates that the test compound can decrease IOP. Exemplary controls/reference values include the expected aqueous humor turnover rate in the absence of the test compound (e.g., an amount or range of amounts of turnover rate expected in the absence of treatment with the test compound).

### C. High-Throughput Screening Methods

In one convenient embodiment, high-throughput screening methods involve providing a combinatorial chemical or peptide library containing a large number of potential therapeutic compounds (e.g., Best2 modulators). Such combinatorial libraries are then screened in one or more assays as described herein to identify those library members (particularly chemical species or subclasses) that display a desired characteristic activity (such as inhibiting or decreasing Best2 activity, such as by increasing intracellular calcium concentration or intracellular pH or decreasing NHE activity or TEP). The compounds thus identified can serve as conventional “lead compounds” or can themselves be used as potential or actual therapeutics. In some instances, pools of candidate compounds may be identified and further screened to determine which individual or subpools of compounds in the collective have a desired activity.

In some cell-based method embodiments described here and throughout the specification, test cells or test compounds can be presented in a manner suitable for high-throughput screening; for example, one or a plurality of test cells (such as cells expressing Best2, such as NPE cells or colon epithelial cells) can be seeded into wells of a microtitre plate, and one or a plurality of test compounds can be added to the wells of the microtitre plate. Alternatively, one or a plurality of test compounds can be presented in a high-throughput format, such as in wells of microtitre plate (either in solution or adhered to the surface of the plate), and contacted with one or a plurality of test cells under conditions that, at least, sustain the test cells. Test compounds can be added to test cells at any concentration that is not toxic to the cells. It is expected that different test compounds will have different effective concentrations. Thus, in some methods, it is advantageous to test a range of test compound concentrations.

### IV. Test Compounds

The methods disclosed herein are of use for identifying compounds that can be used to significantly modulate the biological activity of Best2, such as Best2 modulation of intracellular calcium, pHᵢ, NHE activity and/or TEP. Compounds identified that significantly reduce or inhibit Best2 can in some examples also significantly reduce intraocular pressure.

A “compound” is any substance or any combination of substances that is useful for achieving an end or result. The compounds identified using the methods disclosed herein can be used to modulate (for example, increase or decrease) the biological activity (e.g., regulation of intracellular calcium, pHᵢ, NHE activity and/or TEP) of Best2, and can be used to reduce intraocular pressure. Any compound that has potential (whether or not ultimately realized) to alter Best2 activity can be tested using the methods of this disclosure.

Exemplary test compounds that can be screened for their ability to modulate Best2 activity include, but are not limited to, peptides such as, for example, soluble peptides, including but not limited to members of random peptide libraries (see, e.g., Lam et al., Nature, 354:82-84, 1991; Houghten et al., Nature, 354:84-86, 1991), and combinatorial chemistry-derived molecular libraries made of D- and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), small organic or inorganic molecules (such as, so-called natural products or members of chemical combinatorial libraries), molecular complexes (such as protein complexes), or nucleic acids (e.g., siRNAs). In some examples, the compound is membrane permeable.

Sources of test compounds that can be screened using the disclosed methods include commercial sources (e.g., commercial peptide libraries), as well as molecules generated using routine methods (e.g., antibodies, RNAi molecules). For example, peptide-based diagnostic specific binding molecules that are not necessarily immunoglobulin in origin can be made using methods that are similar to phage display methods. One such method is described in Szardenings, J. Recept. Signal Transduct. Res., 23:307-309, 2003.

Libraries (such as combinatorial chemical libraries) useful in the disclosed methods include, but are not limited to, peptide libraries (see, e.g., U.S. Pat. No. 5,010,175; Furka, Int. J. Pept. Prot. Res., 37:487-493, 1991; Houghton et al., Nature, 354:84-88, 1991; PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA, 90:6909-6913, 1993), vinylogous polypeptides (Hagihara et al., J. Am. Chem. Soc., 114:6568, 1992), nonpeptidal peptidomimetics with glucose scaffolding (Hirschmann et al., J. Am. Chem. Soc., 114:9217-9218, 1992), analogous organic syntheses of small compound libraries (Chen et al., J. Am. Chem. Soc., 116:2661, 1994), oligocarbamates (Cho et al., Science, 261:1303, 1003), and/or peptidyl phosphonates (Campbell et al., J. Org. Chem., 59:658, 1994), nucleic acid libraries (see Sambrook et al. Molecular Cloning, A Laboratory Manual, Cold Springs Harbor Press, N.Y., 1989; Ausubel et al., Current Protocols in Molecular Biology, Green Publishing Associates and Wiley Interscience, N.Y., 1989), peptide nucleic acid libraries (see, e.g., U.S. Pat. No. 5,539,083), antibody libraries (see, e.g., Vaughn et al., Nat. Biotechnol., 14:309-314, 1996; PCT App. No. PCT/US96/10287), carbohydrate libraries (see, e.g., Liang et al., Science, 274:1520-1522, 1996; U.S. Pat. No. 5,593,853), small organic molecule libraries (see, e.g., benzodiazepines, Baum, C&EN, January 18, page 33, 1993, U.S. Pat. No. 5,288,514; isoprenoids, U.S. Pat. No. 5,569,588; thiazolidionones and methathiazones, U.S. Pat. No. 5,549,974; pyrrolidines, U.S. Pat. Nos. 5,525,735 and 5,519,134; morpholino compounds, U.S. Pat. No. 5,506,337) and the like. Additionally, a library of chemical compounds can be obtained, for example from Millennium Pharmaceuticals, Inc. or Celgene Corporation.

Libraries useful for the disclosed screening methods can be produced in a variety of manners including, but not limited to, spatially arrayed multipin peptide synthesis (Geysen, et al., Proc. Natl. Acad. Sci., 81(13):3998-4002, 1984), “tea bag” peptide synthesis (Houghten, Proc. Natl. Acad. Sci., 82(15):5131-5135, 1985), phage display (Scott and Smith, Science, 249:386-390, 1990), spot or disc synthesis (Dittrich et al., Bioorg. Med. Chem. Lett., 8(17):2351-2356, 1998), or split and mix solid phase synthesis on beads (Furka et al., Int. J. Pept. Protein Res., 37(6):487-493, 1991; Lam et a., Chem. Rev., 97(2):411-448, 1997). Libraries may include a varying number of compositions (members), such as up to about 100 members, such as up to about 1000 members, such as up to about 5000 members, such as up to about 10,000 members, such as up to about 100,000 members, such as up to about 500,000 members, or even more than 500,000 members.

In one convenient embodiment, high-throughput screening methods involve providing a combinatorial chemical or peptide library containing a large number of potential therapeutic compounds. Such combinatorial libraries are then screened in one or more assays as described herein to identify those library members (particularly chemical species or subclasses) that display a desired characteristic activity (such as modulating Best2 activity, for example Best2 regulation of intracellular, pHᵢ, NHE activity and/or TEP).

The compounds identified using the methods disclosed herein can serve as conventional “lead compounds” or can themselves be used as potential or actual therapeutics. In some instances, pools of candidate compounds may be identified and further screened to determine which individual or subpools of compounds in the collective have a desired activity.

In one example, the test compound is an antibody. The term “antibody” refers to an immunoglobulin molecule (or combinations thereof) that specifically binds to, or is immunologically reactive with, a particular antigen, and includes polyclonal, monoclonal, genetically engineered and otherwise modified forms of antibodies, including but not limited to chimeric antibodies, humanized antibodies, heteroconjugate antibodies (e.g., bispecific antibodies, diabodies, triabodies, and tetrabodies), single chain Fv antibodies (scFv), polypeptides that contain at least a portion of an immunoglobulin that is sufficient to confer specific antigen binding to the polypeptide, and antigen binding fragments of antibodies. Antibody fragments include proteolytic antibody fragments [such as F(ab′)2 fragments, Fab′ fragments, Fab′-SH fragments, Fab fragments, Fv, and rIgG], recombinant antibody fragments (such as sFv fragments, dsFv fragments, bispecific sFv fragments, bispecific dsFv fragments, diabodies, and triabodies), complementarity determining region (CDR) fragments, camelid antibodies (see, for example, U.S. Pat. Nos. 6,015,695; 6,005,079; 5,874,541; 5,840,526; 5,800,988; and 5,759,808), and antibodies produced by cartilaginous and bony fishes and isolated binding domains thereof (see, for example, International Patent Application No. WO03014161).

A Fab fragment is a monovalent fragment consisting of the VL, VH, CL and CHI domains; a F(ab′)₂ fragment is a bivalent fragment comprising two Fab fragments linked by a disulfide bridge at the hinge region; an Fd fragment consists of the VH and CHI domains; an Fv fragment consists of the VL and VH domains of a single arm of an antibody; and a dAb fragment consists of a VH domain (see, e.g., Ward et al., Nature 341:544-546, 1989). A single-chain antibody (scFv) is an antibody in which a VL and VH region are paired to form a monovalent molecule via a synthetic linker that enables them to be made as a single protein chain (see, e.g., Bird et al., Science, 242: 423-426, 1988; Huston et al., Proc. Natl. Acad. Sci. USA, 85:5879-5883, 1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger et al., Proc. Natl. Acad. Sci. USA, 90:6444-6448, 1993; Poljak et al., Structure, 2:1121-1123, 1994). A chimeric antibody is an antibody that contains one or more regions from one antibody and one or more regions from one or more other antibodies. An antibody may have one or more binding sites. If there is more than one binding site, the binding sites may be identical to one another or may be different. For instance, a naturally occurring immunoglobulin has two identical binding sites, a single-chain antibody or Fab fragment has one binding site, while a “bispecific” or “bifunctional” antibody has two different binding sites.

In some examples, antibodies that specifically bind to a Best2 protein with a binding constant that is at least 10³ M⁻¹ greater, 10⁴ M⁻¹ greater or 10⁵ M⁻¹ greater than a binding constant for other molecules in a sample are screened for their ability to substantially modulate Best2 activity utilizing the methods described herein. In some examples, such an antibody (e.g., monoclonal antibody) or fragment thereof has an equilibrium constant (Kd) of 1 nM or less. In some examples, a Best2 antibody (e.g., monoclonal antibody) or fragment thereof has a binding affinity of at least about 0.1×10⁻⁸ M, at least about 0.3×10⁻⁸ M, at least about 0.5×10⁻⁸ M, at least about 0.75×10⁻⁸ M, at least about 1.0×10⁻⁸ M, at least about 1.3×10⁻⁸ M at least about 1.5×10⁻⁸ M, at least about 2.0×10⁻⁸ M. K_{d} values can, for example, be determined by competitive ELISA (enzyme-linked immunosorbent assay) or using a surface-plasmon resonance device such as the Biacore T100, which is available from Biacore, Inc., Piscataway, N.J.

Antibodies or other test compounds optionally can be directly labeled with a detectable moiety. Useful detection agents include fluorescent compounds (including fluorescein, fluorescein isothiocyanate, rhodamine, 5-dimethylamine-1-napthalenesulfonyl chloride, phycoerythrin, lanthanide phosphors, or the cyanine family of dyes (such as Cy-3 or Cy-5) and the like); bioluminescent compounds (such as luciferase, green fluorescent protein (GFP), or yellow fluorescent protein); enzymes that can produce a detectable reaction product (such as horseradish peroxidase, β-galactosidase, luciferase, alkaline phosphatase, or glucose oxidase and the like), or radiolabels (such as ³H, ¹⁴C, ¹⁵N, ³⁵S ⁹⁰Y, ⁹⁹Tc, ¹¹¹In, ¹²⁵I, or ¹³¹I).

Methods of generating antibodies (such as monoclonal or polyclonal antibodies) are well established in the art (for example, see Harlow and Lane, Antibodies: A Laboratory Manual, Cold Spring Harbor Laboratory, New York, 1988). For example fragments of Best2 can be conjugated to carrier molecules (or nucleic acids encoding such epitopes or conjugated fragments) and can be injected into non-human mammals (such as mice or rabbits), followed by boost injections, to produce an antibody response. Serum isolated from immunized animals may be isolated for the polyclonal antibodies contained therein, or spleens from immunized animals may be used for the production of hybridomas and monoclonal antibodies. In some examples, antibodies are purified before use. In addition, protocols for producing humanized forms of monoclonal antibodies and fragments of monoclonal antibodies are known in the art (see, e.g., U.S. Pat. Nos. 6,054,297, 6,407,213, 6,639,055, 6,800,738, and 6,719,971 and U.S. Pat. Appl. Pub. Nos. 2005/0033031, and 2004/0236078). Similarly, methods for producing single chain antibodies have been described and can be useful for making test compounds (see, Buchner et al., Anal. Biochem. 205:263-270, 1992; Pluckthun, Biotechnology 9:545, 1991; Huse et al., Science 246:1275, 1989 and Ward et al., Nature 341:544, 1989).

In some examples, the test compound is an inhibitory RNA (RNAi) specific for a Best2 nucleic acid sequence. Exemplary RNAi molecules include siRNAs and shRNAs. Such molecules can decrease or eliminate the biological activity of Best2, for example by decreasing translation of Best2. One of ordinary skill in the art can readily generate siRNAs, which specifically bind to a nucleic acid encoding Best2. As described herein, such nucleic acid sequences are publicly available. In an example, commercially available kits, such as siRNA molecule synthesizing kits from PROMEGA® (Madison, Wis.) or AMBION® (Austin, Tex.) may be used to synthesize siRNA molecules. In another example, siRNAs are obtained from commercial sources, such as from QIAGEN® Inc (Germantown, Md.), INVITROGEN® (Carlsbad, Calif.), AMBION (Austin, Tex.), DHARMACON® (Lafayette, Colo.), SIGMA-ALDRICH® (Saint Louis, Mo.) or OPENBIOSYSTEMS® (Huntsville, Ala.).

siRNAs are double-stranded RNAs (dsRNAs) that can induce gene-specific inhibition of expression. These RNAs are suitable for interference or inhibition of expression of Best2 and comprise double-stranded RNAs of about 15 to about 40 nucleotides (such as 19 to 23 nucleotides) containing a 3′ and/or 5′ overhang on each strand having a length of 0- to about 5-nucleotides, wherein the sequence of the double-stranded RNA is substantially identical to a portion of a mRNA or transcript of Best2 for which interference or inhibition of expression is desired. Best2 nucleic acid sequences are known in the art (as described above). The double-stranded RNAs can be formed from complementary ssRNAs or from a single stranded RNA that forms a hairpin or from expression from a DNA vector.

### V. Cells

Cells that can be used in the disclosed methods include cells that express Best2 or functional fragments thereof, such as non-pigmented epithelium or colon epithelium cells that express mammalian Best2. Best2 can be endogenous to the cell or exogenous to the cell (e.g., expressed from a recombinant nucleic acid encoding the protein). In particular examples, the cell is a human, rat, mouse, or pig cell, such as a human, rat, mouse, or pig NPE, salivary acinar, or colon epithelial cell.

In some examples, such cells are primary cells (e.g., directly isolated from a mammalian subject, such as a human or veterinary subject), such as NPE cells or colon epithelial cells. In other examples, such cells are NPE cell lines, such as ODM-2 cells (Marin-Vasallo et al., J. Cell. Physiol. 141:243-252, 1989) or HCE cells (Carre et al., Am. J. Physiol. 273:C1354-C1361, 1997). In additional examples, the cells are colon-derived cell lines, which may express Best2, such as HT-29 cells (ATCC catalog number HTB-38).

In some examples, the cell has substantially no endogenous Best2. Cells expressing exogenous Best2 can be, for example, transiently or stably transfected with an expression vector encoding a Best2 polypeptide (e.g., using publicly available Best2 nucleic acid coding sequences, such as GENBANK Accession Nos. NM_{—}017682.2 (May 1, 2008), AF440756 (Jul. 12, 2002), and AY515705 (Jan. 28, 2004) (human); NM_{—}145388.3 (Jul. 20, 2008), AY450428 (Jun. 24, 2004), and BC019528 (Oct. 27, 2006) (mouse); and NM_{—}001108895 (Apr. 25, 2008) (rat)). One skilled in the art will appreciate that Best2 nucleic acid and protein molecules can vary from those publicly available, such as Best2 sequences having one or more substitutions (for example conservative substitutions), deletions, insertions, or combinations thereof, while still retaining Best2 biological activity. In addition, Best2 molecules include fragments that retain the desired Best2 biological activity.

The present disclosure is illustrated by the following non-limiting Examples.

### EXAMPLES

### Example 1

### Characterization of Bestrophin2 Deficient Mice

This example describes the initial characterization of the phenotype of Best2 deficient mice, including intraocular pressure and aqueous humor dynamics.

### Methods

Targeted Disruption of Mouse Best2 Gene: Best2 deficient mice were generated as described in Bakall et al., Invest. Ophthalmol. Vis. Sci. 49:1563-1570, 2008. Briefly, the targeting vector was constructed using 0.8-kb (5′) and 2.9-kb (3′) mouse Best2 genomic DNA fragments as homology arms. The two arms flanked a promoterless lacZ and a neomycin-resistant gene cassette (lacZ-neo). Homologous recombination in mouse embryonic stem cells resulted in the insertion of the lacZ-neo cassette, replacing a region spanning exon 1 through a part of exon 3 of the mouse Best2 locus. Germ-line-transmitting chimeric mice generated from the targeted embryonic stem cells were bred with C57BL/6 mice to produce Best2^{-/-} mice (Deltagen, San Mateo, Calif.). Intercrossing of heterozygous mice generated Best2^{-/-} mice. Southern blot analysis was performed for identification of homologous recombinants on genomic DNA digested with EcoRV, separated on an 0.8% agarose gel (SeaKem Gold; Cambrex, East Rutherford, N.J.) and transferred to a nylon membrane (Hybond-N⁺; GE Healthcare, Piscataway, N.J.) by capillary blotting. The membrane was hybridized with a 3′ external probe located outside of the homologous arm regions.

IOP Measurements: IOP was measured in the mice by cannulation of the anterior chamber with Avertin® anesthesia (300 mg/kg injected intraperitoneally) as described in, e.g., Husain et al., Exp. Eye Res. 83:1453-1458, 2006; John et al., Invest. Ophthalmol. Vis. Sci. 38:249-253, 1997; Aihara et al., Invest. Ophthalmol. Vis. Sci. 44:5168-5173, 2003. In brief, the anterior chamber was cannulated with a borosilicate glass microneedle (1-mm outer diameter with a 50-μm inner diameter tip, beveled to 45°; Humagen, Charlottesville, Va.) filled with Hanks' balanced salt solution (HBSS) and connected to a pressure transducer (BLPR; World Precision Instruments, Sarasota, Fla.). The signal was amplified (Bridge8 amplifier; World Precision Instruments), converted from analog to digital (iWorx® model 108 converter; CB Sciences, Dover, N.H.), and recorded (LabScribe software ver. 1.6; CB Sciences, Dover, N.H.). IOP was recorded for a period of >90 seconds, and the IOP value determined from the average of each recording. Recordings were discarded if the variance during the recording period exceeded 1 mm Hg or did not immediately return to zero after withdrawal of the needle. All measurements were performed between 2 and 6 PM to avoid diurnal pressure variations. The apparatus was calibrated using a fluid reservoir the height of which could be adjusted to generate a series of known pressures. For experiments using timolol (0.5% solution; Bausch & Lomb, Tampa, Fla.) or brinzolamide (Azopt® 1% solution; Alcon, Fort Worth, Tex.), 10 μL of solution was administrated topically to each eye 2 hours before cannulation.

### Results

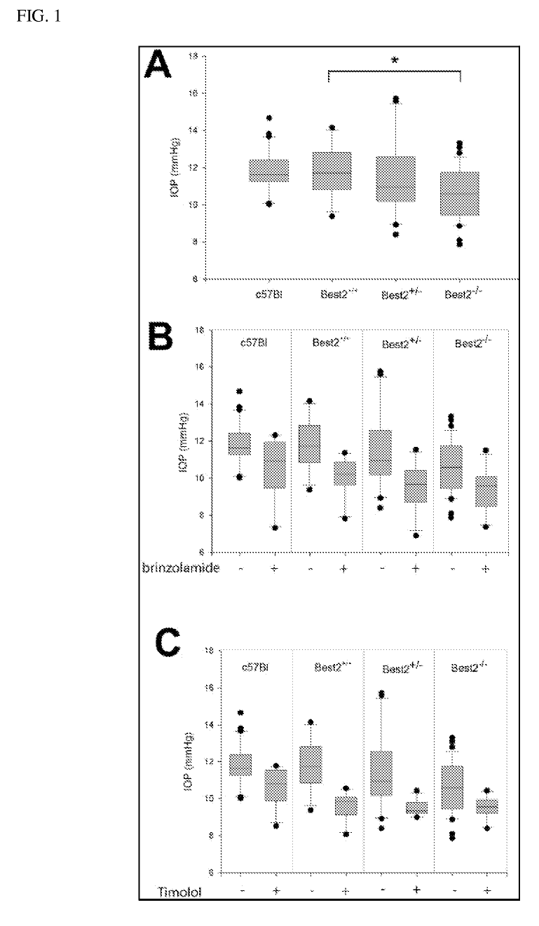

The Best2^{+/+} and Best2^{±} littermates exhibited comparable IOP that did not differ significantly (P=0.979 and P=0.989, respectively) from the C57BL/6 control group (FIG. 1A, Table 1), although there was more variability in the Best2^{±} mice. In contrast, the Best2^{-/-} mice exhibited significantly (P<0.02) lower IOP than did C57BL/6 mice or their Best2^{+/+} and Best2^{±} littermates (FIG. 1A, Table 1).

Mice were treated with either the carbonic anhydrase inhibitor brinzolamide or the β-blocker timolol. Timolol has been shown to be effective in lowering IOP in mice, as have carbonic anhydrase inhibitors (Avila et al., Invest. Ophthalmol. Vis. Sci. 42:1841-1846, 2001) and both drugs are currently used to lower IOP in humans with glaucoma. Brinzolamide was effective in lowering IOP in all mice tested (FIG. 1B, Table 1). Treatment with brinzolamide resulted in reduction to a lower IOP in the Best2^{+/+} and Best2^{-/-} mice than in the C57BL/6 or Best2^{+/+} mice. Brinzolamide was more effective in reducing IOP in the Best2^{+/+} mice, reducing the overall pressure to the same level as observed in the Best2^{-/-} mice treated with the drug (FIG. 1B, Table 1); approximately 1 mm Hg lower than was observed in the C57BL/6 or Best2^{+/+} mice. In contrast, timolol, which lowered IOP significantly (P<0.005) in the C57BL/6 and Best2^{+/+} mice, brought the IOP to the same level in the Best2^{+/+} and Best2^{-/-} mice (FIG. 1C, Table 1), making it less effective in lowering IOP in the Best2^{-/-} mice.

TABLE 1
Mean IOP in Control and Drug-Treated Best2 Mice

| | C57BL/6 | Best2^{+/+} | Best2^{+/-} | Best2^{-/-} |
|---|---|---|---|---|
| Untreated | 11.78 ± 0.22 | 11.73 ± 0.37 | 11.52 ± 0.44 | 10.56 ± 0.21 |
| | (n = 28) | (n = 12) | (n = 20) | (n = 39) |
| Brinzo- | 10.51 ± 0.51 | 10.08 ± 0.33 | 9.56 ± 0.37 | 9.34 ± 0.34 |
| lamide | (n = 11) | (n = 10) | (n = 12) | (n = 11) |
| Timolol | 10.53 ± 0.29 | 9.57 ± 0.22 | 9.48 ± 0.12 | 9.49 ± 0.18 |
| | (n-10) | (n = 11) | (n = 11) | (n = 11) |

Data are mean ± SE (mm Hg)

### Example 2

### Intraocular Pressure and Aqueous Humor Dynamics in Best2 Deficient Mice

This example describes a detailed analysis of the phenotype of bestrophin 2 deficient mice, such as intraocular pressure and aqueous humor dynamics.

Bestrophins are a recently recognized family of proteins that have gained significant interest as potential Ca²⁺ activated chloride channels (CaCCs), although this function is controversial (Marmorstein et al., Exp. Eye Res. 85:423-424, 2007; Hartzell et al., Physiol. Rev. 88:639-672, 2008). Recently, mice in which the gene Best2, encoding bestrophin-2 (Best2) was disrupted were shown to exhibit a significantly lower IOP than Best2^{+/+} littermates (Example 1; Bakall et al., Invest. Ophthalmol. Vis. Sci. 49:1563-1570, 2008). Best2 is uniquely expressed in the basolateral plasma membrane of the NPE in the eye, as well as in several other tissues including the transporting epithelium of the colon.

A comprehensive study of aqueous dynamics in Best2^{-/-} mice was performed. Although IOP in these mice is lower in comparison to Best^{+/+} mice, the rate of aqueous formation, Fₐ, is significantly increased, as is drainage through both the conventional (F_{c}) and uveoscleral (Fᵤ) outflow pathways. Morphologic inspection of the anterior chamber did not identify any developmental or anatomical changes that would explain this phenomenon. Based on these data, Best2 does not participate in, but rather antagonizes, the formation of aqueous humor, and there is a communicative link between the ciliary epithelia and the outflow pathways that involves signaling via the only common component of the two, the aqueous humor. These data are consistent with bestrophins being regulators of ion transport.

### Methods

IOP Measurements: IOP was measured in Best2^{+/+} and Best2^{-/-} mice as described in Example 1.

EVP Measurement: Measurements of EVP (episcleral venous pressure) were performed as described by Aihara et al. (Invest. Ophthalmol. Vis. Sci. 44:5168-5173, 2003). IOP was first measured as described in Example 1, after which the height of the saline-filled reservoir was adjusted to equal the IOP, and the stopcock between the transducer and reservoir was opened. The height of the reservoir was decreased 0.5 mm Hg (0.68 mm H₂O) per minute. EVP was determined as the pressure at which reflux of blood from collector channels into Schlemm's canal could be observed through a dissecting microscope. The reservoir was then returned to its original height, and the measurement repeated in the same eye. Differences in IOP and EVP between experimental groups were compared by t-test.

Determination of Aqueous Flow: The rate of aqueous humor formation was determined by dilution of a fluorescent perfusate according to the method of Aihara and co-workers (Invest. Ophthalmol. Vis. Sci. 44:5168-5173, 2003), substituting 5 μg/ml FITC-Dextran (70 kDa) for rhodamine dextran. Perfusion pressure was maintained at EVP so that pressure-dependent outflow was reduced to zero. Fluorescence was determined on a Wallac Victor3™ 1420 Multilabel Counter (PerkinElmer Life Sciences) with excitation and emission wavelengths of 485 nm and 535 nm, respectively. Aqueous flow (Fₐ) was calculated based on the aspiration rate (3 μl/min) and the ratio of the concentration of FITC-dextran in the perfusion outflow fluid (Cₒ) to the concentration of FITC-dextran in the perfusion inflow fluid (Cᵢ) according to Aihara et al. (Invest. Ophthalmol. Vis. Sci. 44:5168-5173, 2003):

Fₐ=3 μl min⁻¹ (1−Cₒ/Cᵢ) (Equation 1)

Measurement of Conventional Outflow Facility: Conventional outflow facility (Cₜ) was determined according to Aihara et al., (Invest. Ophthalmol. Vis. Sci. 44:5168-5173, 2003) using the same infusion system used to measure IOP and aqueous production. All fluid within the infusion system was replaced with physiological saline. The measurement was based on measuring total outflow volume (Vₜ) for a period of 10 minutes at two different levels of IOP (25 and 35 mm Hg) maintained by altering the reservoir height. Cₜ was determined according to the following equation:

Cₜ=0.01×(Vₜ₌₃₅−Vₜ₌₂₅) μl min⁻¹/mm Hg (Equation 2)

Determination of Conventional and Uveoscleral Outflow: IOP, EVP, and Ct were measured and averaged for each genotype. Using these data, the conventional outflow (F_{c}) was calculated as:

F_{c}=Cₜ×(IOP−EVP) (Equation 3)

Uveoscleral outflow (Fᵤ) was then calculated according to the modified Goldmann equation:

Fᵤ=Fₐ−Cₜ×(IOP−EVP) (Equation 4)

Anterior chamber volume and aqueous humor turnover rate: Anterior chamber volume (Vₐ) was determined by aspiration of the aqueous humor at a rate of 100 nl/sec, according to Aihara et al., (Invest. Ophthalmol. Vis. Sci. 44:5168-5173, 2003). Aspiration was deemed complete when the central border of the iris was observed under a dissecting microscope to make contact with the cornea. The turnover rate was calculated as:

Turnover rate % min⁻¹=100×Fₐ/Vₐ (Equation 5)

Statistical analysis: Data from Best2^{+/+} and Best2^{-/-} mice were compared using the t-test function (2-tailed, homoscedastic) in Microsoft Excel 2004 for MAC.

Histology: 2-month old mice were fixed by intracardiac perfusion with half-strength Karnovsky's fixative (2.5% gluteraldehyde and 2% paraformaldehyde in 0.1 M cacodylate buffer, pH 7.2). Eyes were enucleated and further fixed by immersion in half strength Karnovsky's fixative for an additional 18 hrs, after which they were transferred to 0.1M cacodylate buffer (pH 7.2). Eyes were then post-fixed with 1% osmium tetroxide, dehydrated in a graded series of alcohols, and embedded in Spurr's resin. Thick sections (0.5 μm) were cut on a Reichert Ultracut microtome and stained with Toluidine blue. Sections were inspected using a Nikon E-600 microscope and photographed using a color CCD camera.

Optical coherence tomography: 2-4 month old mice were anesthetized with Avertin® (250 mg/kg IP) and placed on a mechanical stage that permitted movement along 2 axes. The anterior chamber was imaged using an OCP930SR Spectral Radar OCT Imaging System (Thorlabs, Newton, N.J.) with a 930 nm center wavelength light source and an axial resolution 4.5 μm in tissue. Cross-sectional images were recorded in the nasal-temporal plane as well as the superior-inferior plane. 500×512 (XZ) pixel images were captured at maximum pupillary diameter and corrected for asymmetries in X-Z spacing using Adobe Photoshop 7.01. Measurements of anterior chamber depth and corneal thickness were made from distortion corrected images obtained along both the nasal-temporal and superior-inferior axis using Image J software (Available online from Image J).

### Results

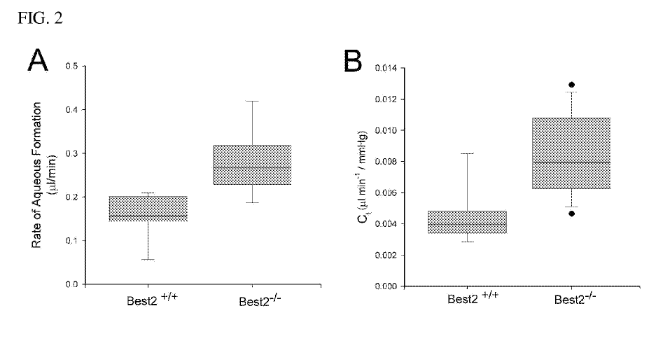

Effect of Best2 disruption on IOP: IOP was measured in Best2^{-/-} mice and their Best2^{+/+} littermates. The measured IOP in Best2^{+/+} mice was 11.70±0.16 mm Hg (mean±SE, n=31) and for Best2^{-/-} it was 10.22±0.16 mm Hg (mean±SE, n=55), a significant (P<0.0001) difference of 1.48 mm Hg (FIG. 2, Table 2).

Measurement of aqueous humor formation: According to the modified Goldmann equation, IOP=[(Fₐ−Fᵤ)/Cₜ]+EVP. EVP was measured in Best2^{+/+} and Best2^{-/-} mice and no difference was found, with both groups having an EVP of 6.3±0.3 mm Hg (Table 2). Since EVP values between Best2^{+/+} and Best2^{-/-} mice are identical, the difference in IOP must arise from differences in Fₐ or Cₜ. The rate of aqueous flow (Fₐ) was determined by a modification of the method of Aihara et al. (Invest. Ophthalmol. Vis. Sci. 44:5168-5173, 2003) in which the anterior chamber of the eye was cannulated, clamped at EVP, and perfused with a balanced salt solution containing a 50 kDa FITC Dextran. By following dilution of the fluorescence emission of the perfusion outflow fluid versus that of the inflow fluid, Fₐ was be determined. In Best2^{+/+} mice Fₐ was 0.160±0.047 μl/min (mean±SD, n=9) (FIG. 2A, Table 2), similar to the 0.18±0.05 μl/min reported by Aihara et al. (Invest. Ophthalmol. Vis. Sci. 44:5168-5173, 2003) for NIH Swiss white mice. Unexpectedly, Fₐ for Best2^{-/-} mice was 0.277±0.069 μl/min (mean±SD, n=9) (FIG. 2A, Table 2), a significant (P<0.001) increase of 73% over the Best2^{+/+} mice.

Measurement of conventional outflow facility: Based on the modified Goldmann equation (Equation 4), for IOP to be diminished without altering EVP, either Fₐ must be diminished or Fᵤ and Cₜ must be increased. Since Fₐ was significantly increased for Best2^{-/-} mice, and Fᵤ can not be measured directly, Cₜ was measured (FIG. 2B, Table 2).

Again employing the methods of Aihara et al. (Invest. Ophthalmol. Vis. Sci. 44:5168-5173, 2003) Cₜ was determined in Best2^{+/+} mice to be 0.0050±0.0015 μl min⁻¹/mm Hg (mean±SD, n=23, FIG. 2B, Table 2), nearly identical to the 0.0051 μl min⁻¹/mm Hg value reported by Aihara et al. (Invest. Ophthalmol. Vis. Sci. 44:5168-5173, 2003) for NIH Swiss white mice. For Best2^{-/-} mice, Cₜ was 0.0085±0.0026 μl min⁻¹/mm Hg (mean±SD, n=23, FIG. 2B, Table 2), a significant (P<0.001) increase of 70% compared to Best2^{+/+} mice.

Having obtained measured values for IOP, EVP, Fₐ, and Cₜ, F_{c} and Fᵤ were calculated using Equations 3 and 4 (see Methods), respectively. As shown in Table 2, F_{c} was 0.027 μl/min and 0.033 μl/min in Best2^{+/+} and Best2^{-/-} mice, respectively, a 22% increase in F_{c} for Best2^{-/-} mice. Fᵤ was calculated to be 0.133 μl/min and 0.211 μl/min in Best2^{+/+} and Best2^{-/-} mice, respectively, a 59% increase in Best2^{-/-} mice. These results indicate that both pressure-dependent and pressure-independent outflow appears to overcompensate for the increase in Fₐ in Best2^{-/-} mice, resulting in an IOP that is lower in Best2^{-/-} mice than in Best2^{+/+} mice despite the 73% increase in Fa in Best2^{-/-} mice.

TABLE 2
Aqueous Humor Dynamic Parameters of Best2^{+/+} and Best2^{-/-} Mice

| | Best2^{+/+} | | | Best2^{-/-} | | |
|---|---|---|---|---|---|---|
| Parameter | Data | n | Equation | Data | n | Equation |
| IOP (mm Hg) | 11.70 ± 0.16 | 31 | | 10.22 ± 0.16 | 55 | |
| EVP (mm Hg) | 6.3 ± 0.3 | 9 | | 6.3 ± 0.3 | 9 | |
| Fₐ (μl/min) | 0.160 ± 0.047 | 9 | 1 | 0.277 ± 0.069 | 9 | 1 |
| C (μl min⁻¹/mm Hg) | 0.0050 ± 0.0015 | 23 | 2 | 0.0085 ± 0.0026 | 23 | 2 |
| F_{c} (μl/min) | 0.027 | | 3 | 0.033 | | 3 |
| Fᵤ (μl/min) | 0.133 | | 4 | 0.244 | | 4 |
| Vₐ (μl) | 4.51 ± 0.37 | 8 | | 3.74 ± 0.27 | 12 | |
| Turnover rate | 3.5 | | 5 | 7.4 | | 5 |
| (%/min) | | | | | | |

Data are presented as Mean ± SD except for IOP, where data are Mean ± SE
Values for F_{c}, Fᵤ, and turnover rate were derived from measured data using the indicated equations (see Methods)

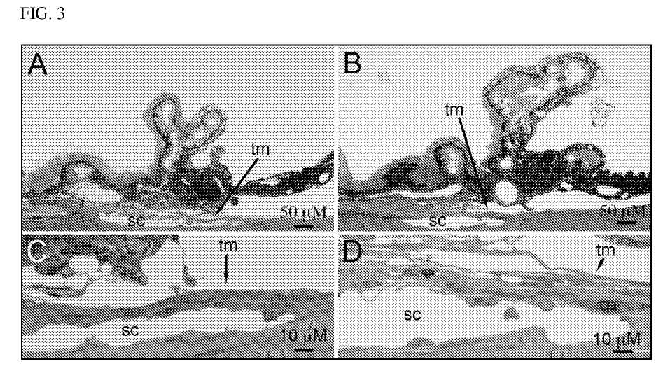

Anatomy of the angle: A post-mortem examination of the angles of Best2^{+/+} and Best2^{-/-} mice identified no gross anatomical abnormalities to outflow structures; however, an expanded trabecular meshwork (TM) and deposits of pigment in the TM of Best2^{-/-} mice were occasionally observed (FIG. 3).

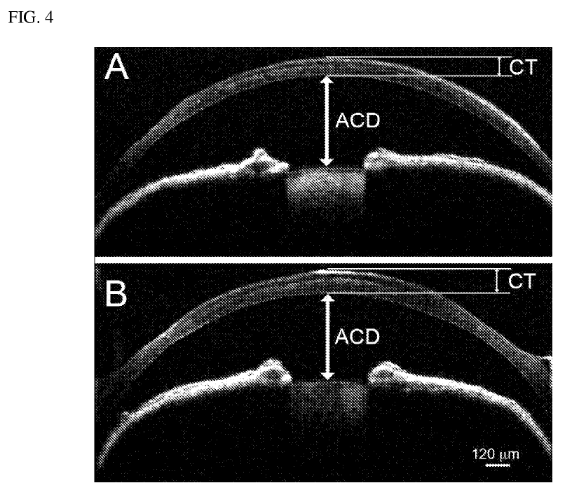

In parallel studies, the anterior chamber of live mice was examined using OCT (FIG. 4). No difference was observed in the thickness of cornea (Table 3). However, the anterior chamber depth (ACD) measured from the inner surface of the cornea to the anterior surface of the lens capsule was 496 μm in Best2^{+/+} mice (n=12) but only 458 μm in Best2^{-/-} mice (n=15), a decrease of nearly 40 μm (FIG. 4, Table 3).

To determine anterior chamber volume (Vₐ) the aqueous humor aspiration method of Aihara et al. (Invest. Ophthalmol. Vis. Sci. 44:5168-5173, 2003) was used. Consistent with the change observed in ACD, the anterior chamber volume was decreased by 17% in Best2^{-/-} mice (Table 2). Based on Vₐ and Fₐ, the turnover rate of aqueous humor was calculated using Equation 5 (see methods). Turnover in Best2^{+/+} and Best2^{+/+} mice was 3.5 % min⁻¹ and 7.4% min⁻¹ respectively (Table 2); a 2.1-fold increase in the Best2^{-/-} mice.

TABLE 3
Anterior Chamber Structural Attributes of
Best2^{+/+} and Best2^{-/-} Mice

| | Anterior Chamber Depth (μm) | Corneal Thickness (μm) |
|---|---|---|
| Best2^{+/+} | 496 ± 27 | 100 ± 16 |
| Best2^{-/-} | 458 ± 17* | 101 ± 13 |

Data are Mean ± SD;
Best2^{+/+} n = 12,
Best2^{-/-} n = 15
*p < 0.001

### Discussion

This study demonstrated that Best2^{-/-} mice exhibit a diminished IOP despite a ˜73% increase in Fₐ and a >2-fold increase in the rate of aqueous turnover. The increase in Fₐ was overcompensated for by an enhanced drainage. Both F_{c} and Fᵤ were increased, although a more significant portion of the aqueous flow drained via Fᵤ than F_{c} in the Best2^{-/-} mice. Based on the antagonistic effect of Best2 on Fₐ it appears that Best2 is not one of the Cl⁻ channels involved in aqueous humor formation.

The increase in Fₐ observed in Best2^{-/-} mice in the present study should logically have led to an increase in IOP. However, there was an overcompensating effect on the conventional outflow facility that resulted in a diminished IOP. No obvious anatomical changes, such as breaks in the inner wall of Schlemm's canal or larger spaces between ciliary muscle bundles were observed that would account for elevated drainage through either the conventional or uveoscleral pathways. However, TM tissues that appeared expanded were occasionally observed, perhaps the result of enhanced outflow. Such a difference may be more obvious if eyes are fixed under pressure. If there is no dramatic developmental/structural abnormality in the angle, then the increase in Cₜ must be functional and have been triggered by the increase in Fₐ.

In summary, we have shown that disruption of Best2 in mice results in a significant decrease in IOP despite a 73% increase in Fₐ. Therefore, Best2 is a potent antagonist of aqueous humor production. The enhanced production of aqueous humor is overcompensated for by an increase in both conventional and uveoscleral drainage. Since Best2 is expressed uniquely in the NPE, and there is no obvious anatomic reason for increased outflow, these data imply a communicative link between the ciliary body and the outflow pathway that is modulated by Best2.

### Example 3

### Regulation of Intracellular pH by Bestrophins

This example describes regulation of intracellular pH and Na⁺/H⁺ exchange by bestrophins.

### Methods

The pH indicator seminaphtorhodafluor-1 (SNARF-1) was used to determine pHᵢ. Mouse tissue preparations or cultured cells on glass coverslips were loaded with SNARF-1 carboxylic acid and then secured in a perfusion chamber on the stage of a Nikon TE200 inverted microscope. The chamber was continuously perfused with BSS (125 mM NaCl, 5 mM KCl, 1.2 mM MgSO₄, 1 mM CaCl₂, 2 mM KH₂PO₄, 5 mM glucose, 32.2 mM HEPES, pH 7.4) warmed to 37° C. by an in-line heater. In ion substitution experiments, Na⁺ was replaced using N-methyl-D-glucamine (NMDG). Acid loading of cells was accomplished by addition of 20 mM NH₄Cl to BSS. SNARF-1 fluorescence excited with 540 nm light was imaged at 10 second intervals through a 40× air or oil objective using a Cascade 512b cooled CCD camera (Photometrics, USA) and a Dual-view imager (Optical Insights, LLC, Tucson Ariz.) to permit simultaneous acquisition of SNARF-1 emissions using 580/20 nm and 640/20 nm bandpass filters. Data were collected and the intensities and ratios of emissions at the two ion sensitive wavelengths determined using MetaFluor Software (Molecular Devices, USA). All experiments were calibrated using the nigericin-high K⁺ technique (Thomas et al., Biochemistry 18:2210-2218, 1979).

HEK293 cells were transfected to express hBest1, mBest2, or the hBest1 mutants W93C or R218C. To verify expression of hBest1 or mBest2 in transfected cells following imaging experiments, coverslips were immersed in −20° C. methanol, then stained with monoclonal antibody E6-6 for hBest1 (Santa Cruz Biotechnology, Santa Cruz, Calif.) or a rabbit polyclonal antibody recognizing mBest2 as described previously (Bakall et al., Invest. Ophthalmol. Vis. Sci. 49:1563-1570, 2008). Fields were examined to insure expression in the specific grid number that was used. All cells in the grid were used regardless of individual level of expression.

### Results

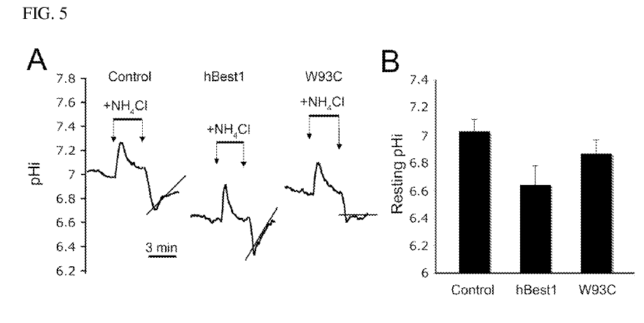

The effects of bestrophins on pHᵢ were determined in human fetal RPE (fhRPE) monolayers using the indicator dye SNARF-1. Experiments performed in Ringer's solution containing CO₂/HCO₃⁻ indicated that Best1 acidified cells and enhanced the ability of the cells to recover from an acid load induced by an NH₄Cl pulse (FIG. 5A). Best1W93C (a Best1 protein with a W93C mutation) inhibited the ability of the cells to recover from an acid load (FIG. 5A). In addition, there was a significant (p<0.01) decrease in the resting pHᵢ of RPE cells overexpressing Best1 or Best1W93C (FIG. 5B) an effect opposite that observed in HCO₃⁻ containing media.

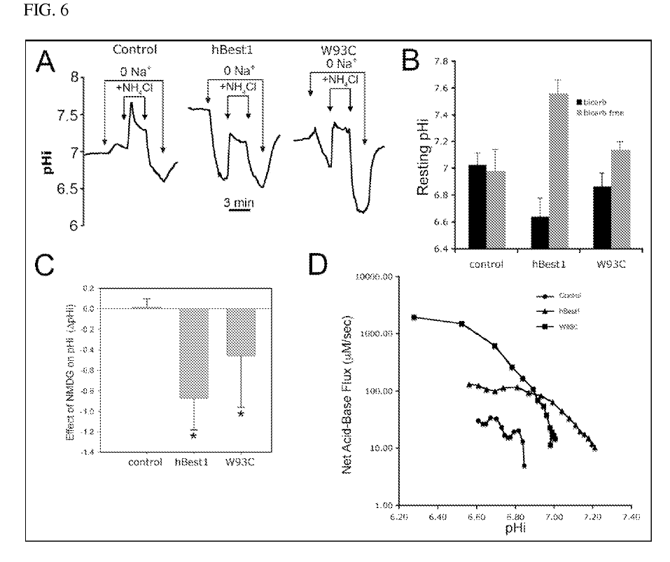

Intracellular pH was determined in fhRPE cells expressing endogenous Best1 (control) or overexpressing hBest1 or the hBest1W93C in nominally HCO₃⁻ free HEPES buffered balanced salt solution. An acid load was induced by addition of 20 mM NH₄Cl. Recovery from the acid load did not occur until Na⁺ was restored (FIG. 6A). As noted above, cells overexpressing hBest1 or W93C had decreased pHᵢ compared to control cells, however, in HCO₃⁻ free buffer, the pHᵢ was increased compared to control cells (FIG. 6B). Substitution of Na⁺ with the organic cation N-methyl-D-glucamine (NMDG) caused a significant decrease in pHᵢ in hBest1 and W93C overexpressing cells (FIG. 6C). The recovery of pHᵢ from the acid load was examined by analysis of net acid base flux (FIG. 6D) according to Boyarsky et al. (Proc. Natl. Acad. Sci. USA 87:5921-5924, 1990; Am. J. Physiol. 255:C857-C869, 1988). W93C had the fastest recovery from an acid load as illustrated by their high net acid-base flux (FIG. 6D). Recovery of hBest1 overexpressing cells was also increased relative to controls.

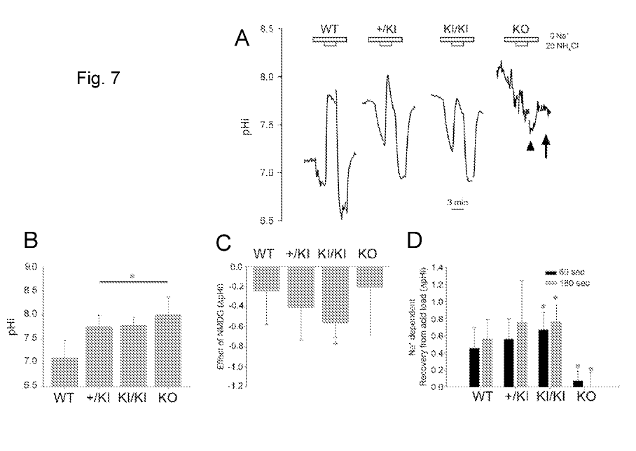

Na⁺/H⁺ exchanger (NHE) activity was measured in RPE cells in eyecup preparations from Best1W93C knockin (KI) mice (FIG. 7). In RPE obtained from Best1^{+/ki} or Best1^{ki/ki} mice, resting pHᵢ was increased significantly (p<0.001) over that of wild type littermate controls (FIG. 7A and 7B). Substitution of Na⁺ with NMDG resulted in a significantly greater decrease in pHᵢ in cells isolated from Best1^{+/ki} and Best1^{ki/ki} mice (p<0.05) than that observed in cells from WT littermates (FIG. 7C). Recovery from an acid load was accelerated in Best1^{+/ki} and Best1^{ki/ki} (p<0.01) mice and exceeded the overall recovery in WT littermates (FIG. 7A and 7D). Conversely, in eyecup preparations from Best1^{-/-} mice pHᵢ was significantly (p<0.05) higher than that observed from WT, Best1^{+/ki} or Best¹^{ki/ki} mice (FIG. 7A and 7B). The acidification of the cells due to substitution of Na⁺ with NMDG (FIG. 7C) was similar to that observed in WT mice, but differed from that observed in Best1^{+/ki} or Best1^{ki/ki} (p<0.01) mice. Interestingly, the cellular alkalinization in the presence of NH₄Cl was substantially smaller in Best1^{-/-} mice than in all other mice and cell cultures tested and the acid load imparted after subsequent removal of NH₄Cl from the medium was also modest in comparison. The small acidification following the NH₄Cl pulse was rapidly reversed in the absence of Na⁺, and restoration of Na⁺ caused little alkalinization indicating a near or total lack of NHE activity (FIG. 7A and 7D) in Best1^{-/-} mice.

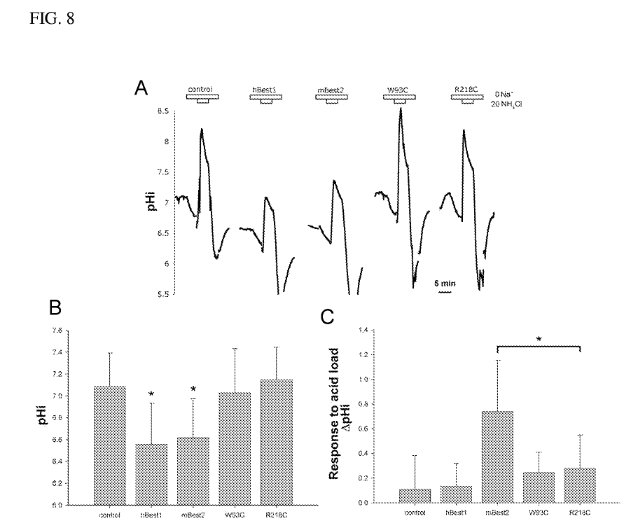

Expression of mBest2 in HEK293 cells also caused a significant decrease in resting pHi, which was similar to the decrease in resting pHᵢ caused by hBest1 expression (FIG. 8A and 8B). A small but equivalent acidification was induced by substitution of Na⁺ with NMDG in all cells (0 Na⁺, FIG. 8A). Induction of an acid load via a 20 mM NH₄Cl pulse resulted in a greater acidification of cells expressing hBest1, mBest2, or mutant hBest1 (FIG. 8A) and a significantly faster recovery (FIG. 8C) in cells expressing mBest2 over the first 60 seconds.

### Example 4

### Regulation of Transepithelial Potential by Bestrophins

This example describes regulation of transepithelial potential (TEP) in RPE cell monolayers by bestrophins.

### Methods

Human fetal RPE (fhRPE) monolayers were grown on Millicell® inserts and mounted in a water jacketed Ussing chamber similar to that previously described (Oakley et al., Invest. Ophthalmol. Vis. Sci. 16:771-774, 1977; Miller and Steinberg, J. Membr. Biol. 36:337-372, 1977). The chamber was modified to hold Millicell® HA cell culture inserts. The TEP was recorded using Ag/AgCl pellet electrodes bridged with agar 0.5 M KCl bridges in each bath. Signals were passed through a low pass Bessel filter, amplified using a DP-304 Differential Amplifier (Warner Instruments, Inc., Hamden, Conn.), digitized, and recorded and analyzed on a Dell PC using LabScribe 1.821 software (iWorx®, Dover, N.H.). The transepithelial resistance (TER) of the monolayers was determined by passing bipolar 10 μA pulses (DS8000 Digital Stimulator, World Precision Instruments, Inc., Sarasota, Fla.) across the monolayer every 30 seconds between Ag—AgCl pellets placed in each bath. TER was measured from the current induced voltage changes across the monolayer. I_{sc} was calculated as I_{sc}=(TEP/TER).

All experiments began by recording TEP with both sides of the chamber filled with Ringer's solution containing: 113.4 mM NaCl, 5 mM KCl, 5.6 mM glucose, 0.8 mM MgCl₂, 2 mM glutathione, 1.8 mM CaCl₂, and 26.2 mM NaHCO₃, pH 7.4, and gassed with 95% O2, 5% CO2. The chamber was continuously perfused with fresh Ringer's solution.

### Results

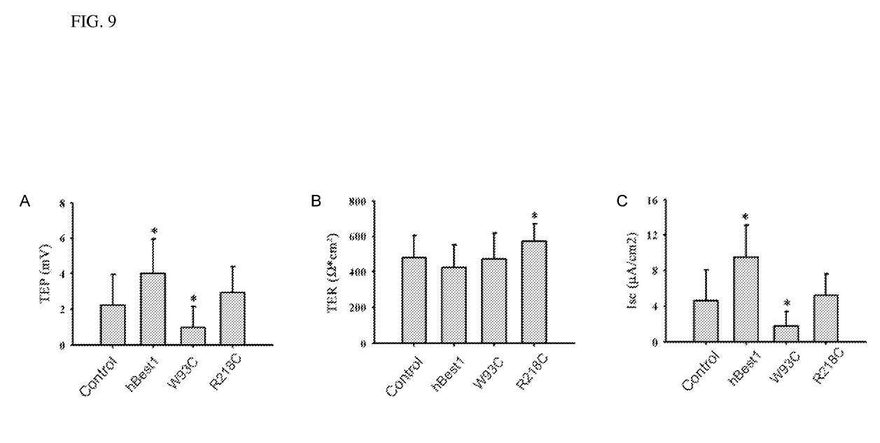

fhRPE monolayers were grown according to the method of Hu and Bok (Mol. Vis. 7:14-19, 2001). These cultures express endogenous Best1. Human Best1 (hBest1) and the hBest1 W93C and R218C mutants were overexpressed in the monolayers using adenovirus mediated gene transfer. Overexpression of hBest1 resulted in a significant (p<0.05) increase in TEP (FIG. 9A), which translated into an increase in the calculated I_{sc} (FIG. 9C). Monolayers expressing hBest1 W93C exhibited a reduced TEP and I_{sc} (FIG. 9A and FIG. 9C). Monolayers expressing hBest1 R218C exhibited a small but significant decrease in TER, but no change in I_{sc} compared to controls (FIG. 9B and FIG. 9C).

### Example 5

### Screening Methods

This example provides exemplary methods for screening compounds for modulators of Best2 activity that decrease IOP.

Based upon the teachings herein, methods for screening compounds capable of modulating Best2 activity are disclosed herein. In a particular example, in vitro assays of intracellular calcium concentration or intracellular pH are performed to identify compounds capable of modulating Best2 activity. In other examples, in vivo assays are utilized to identify Best2 modulators that decrease intraocular pressure.

(1) In vitro Cell-based Screening Assay

A library of chemical compounds is obtained and screened for their effect on intracellular calcium concentration using the assays disclosed. Cells that express Best2 (such as HT-29 cells or HEK293 cells expressing Best2) are loaded with 10 μM Fura-2-AM for 45 minutes. The cells are contacted with the test compound either simultaneously with or following the stimulator of intracellular calcium ATP for 1 hour at 37° C. Absolute values of intracellular free calcium are estimated using intracellular calibration according to Grynkiewicz et al. (J. Biol. Chem. 260:3440-3450, 1985) using bath solution containing 10 μM ionomycin to saturate fura-2 with calcium, and calcium-free bath solution with 10 μM ionomycin to deplete fura-2 from calcium. Changes in intracellular calcium are measured with a fluorometer. An agent that results in at least a 20% increase in intracellular calcium (e.g. at least about 50%, at least about 100%, at least about 200%, at least about 300%, at least about 400%, or at least about 500% increase) is identified as a Best2 inhibitor capable of decreasing IOP.

In some cases, a library of chemical compounds is obtained and screened for their effect on pHᵢ using the assays disclosed. The effect of the compounds on pHᵢ may be assayed instead of, or in addition to, assaying the effect on intracellular calcium. Cells that express Best2 (such as HT-29 cells or HEK293 cells expressing Best2) cultured on glass coverslips are loaded with the pH indicator seminaphtorhodafluor-1 (SNARF-1) and then secured in a perfusion chamber on the stage of a Nikon TE200 inverted microscope. The chamber is continuously perfused with balanced salt solution (125 mM NaCl, 5 mM KCl, 1.2 mM MgSO₄, 1 mM CaCl₂, 2 mM KH₂PO₄, 5 mM glucose, 32.2 mM HEPES, pH 7.4) warmed to 37° C. by an in-line heater. SNARF-1 fluorescence excited with 540 nm light is imaged at 10 second intervals through a 40× air or oil objective using a Cascade 512b cooled CCD camera (Photometrics, USA) and a Dual-view imager (Optical Insights, LLC, Tucson Ariz.) to permit simultaneous acquisition of SNARF-1 emissions using 580/20 nm and 640/20 nm bandpass filters. Data are collected and the intensities and ratios of emissions at the two ion sensitive wavelengths determined using MetaFluor Software (Molecular Devices, USA). All experiments are calibrated using the nigericin-high K⁺ technique (Thomas et al., Biochemistry 18:2210-2218, 1979). An agent that results in at least a 1% increase in pHᵢ (such as an at least 2%, at least 3%, at least 4%, at least 5%, at least 10%, or at least 20% increase) is identified as a Best2 inhibitor capable of decreasing IOP.

The identified compounds are also used as lead compounds to identify other compounds having even greater inhibitory effects on Best2. For example, chemical analogs of identified chemical entities, or variant, fragments, or fusions of peptide agents, are tested for their activity methods described herein. Candidate compounds also can be tested in cell lines and animal models to determine their therapeutic value (as described herein). The compounds also can be tested for safety in animals, and then used for clinical trials in animals or humans.

(2) In vivo Screening Assay

A library of chemical compounds or a compound identified as a candidate in the described in vitro assays is obtained and screened for their effect on IOP using the assays disclosed. Wild type mice (such as C57BL/6 mice) or a mouse model of glaucoma (such as DBA/2J mice) are treated with one or more test compounds (dosage ranging from 0.1 pg/kg to 10 mg/kg). In one example, the test agent is provided by topical administration (such as eye drops). Two hours later, IOP is measured by cannulation of the eye (as described in Example 1) or by tonometry. An at least 5% decrease in IOP as compared to a known value or IOP in the absence of the test agent, identifies the test agent as an inhibitor of Best2.

In view of the many possible embodiments to which the principles of the disclosure may be applied, it should be recognized that the illustrated embodiments are only preferred examples and should not be taken as limiting the scope of the invention. Rather, the scope of the invention is defined by the following claims. I therefore claim as my invention all that comes within the scope and spirit of these claims.

I claim:

1. A method for identifying a compound that modulates bestrophin 2 activity, comprising:
contacting a cell comprising bestrophin 2 with a test compound;
determining a cell characteristic in the presence of the test compound, wherein the cell characteristic is selected from the group consisting of intracellular calcium concentration, intracellular pH, Na⁺/H⁺ exchanger activity, transepithelial potential, and combinations thereof; and
selecting a test compound that alters the cell characteristic in the presence of the test compound as compared to in the absence of the test compound, wherein a compound that alters the cellular characteristic in the presence of the test compound as compared to in the absence of the test compound is a compound that modulates bestrophin 2 activity.

2. The method of claim 1, wherein the selected test compound is a test compound that increases intracellular calcium concentration in the presence of the test compound as compared to in the absence of the test compound.

3. The method of claim 1, further comprising contacting the cell with a second compound that increases intracellular calcium in the absence of the test compound.

4. The method of claim 3, wherein the second compound is selected from the group consisting of adenosine triphosphate, dopamine, epinephrine, and a combination thereof.

5. The method of claim 3, wherein the selected test compound is a test compound that increases intracellular calcium concentration in the presence of the test compound as compared to in the absence of the test compound.

6. The method of claim 1, wherein an increase in intracellular calcium concentration in the presence of the test compound as compared to in the absence of the test compound indicates that the test compound is a compound that decreases intraocular pressure.

7. The method of claim 1, wherein the selected test compound is a test compound that increases intracellular pH in the presence of the test compound as compared to in the absence of the test compound.

8. The method of claim 1, further comprising contacting the cell with a second compound that increases intracellular pH in the absence of the test compound.

9. The method of claim 8, wherein the second compound comprises ammonium chloride.

10. The method of claim 8, wherein the selected test compound is a test compound that increases intracellular pH in the presence of the test compound as compared to in the absence of the test compound.

11. The method of claim 1, wherein a decrease in intracellular pH in the presence of the test compound as compared to in the absence of the test compound indicates that the test compound is a compound that decreases intraocular pressure.

12. The method of claim 1, wherein the cell is a non-pigmented epithelium cell or a colon epithelium cell.

13. The method of claim 1, wherein the cell is in a subject, and contacting the test compound with the cell comprises administering the test compound to the subject.

14. The method of claim 13, further comprising:
determining an ocular phenotype in the subject, wherein the ocular phenotype is selected from the group consisting of intraocular pressure, aqueous flow, outflow facility, turnover of aqueous humor, and combinations thereof; and
selecting a compound that alters the ocular phenotype in the subject as compared to the ocular phenotype in the absence of the test compound, wherein a compound that alters the ocular phenotype in the presence of the test compound as compared to in the absence of the test compound is a compound that modulates bestrophin 2 activity.

15. The method of claim 14, wherein the selected test compound is a test compound that reduces intraocular pressure in the presence of the test compound as compared to in the absence of the test compound.

16. The method of claim 14, wherein the selected test compound is a test compound that increases aqueous flow, outflow facility, and/or turnover of aqueous humor in the presence of the test compound as compared to in the absence of the test compound.

17. A method of identifying a compound that decreases intraocular pressure, comprising:
contacting a cell comprising bestrophin 2 with a test compound; and
measuring intracellular calcium concentration, wherein an increase in intracellular calcium concentration indicates that the test compound is an compound that decreases intraocular pressure.

18. A method of identifying a compound that decreases intraocular pressure, comprising:
contacting a cell comprising bestrophin 2 with a test compound; and
measuring intracellular pH, wherein an increase in intracellular pH indicates that the test compound is an compound that decreases intraocular pressure.


SEQUENCE LISTING (4 sequences)

SEQ ID NO: 1 - DNA, 1924 bp, Homo sapiens
  CDS (1)..(1530)
atgaccgtcacctacacagcccgagtggcgaacgcccgcttcggtggcttctcccagctgctgctactgtggcgtgggagcatctacaaactcctgtggcgagagctgctctgcttccttgggttctacatggcgctgagtgctgcctaccgctttgtgctgaccgaagggcagaagcgctacttcgagaagcttgtgatttattgtgaccagtatgccagcctcatccctgtctccttcgtgcttggcttttatgtgacgctggtggtgaaccgctggtggagccagtacctatgcatgccgctgcccgacgcgctcatgtgcgtggtggcgggcaccgtgcacggacgcgacgaccgcggccgcctctaccggcgcacactcatgcgctacgcagggctctcggccgtgctcatcctgcgctccgtcagcaccgcggtgttcaagcgcttccccaccatagaccacgtggtggaggctgggtttatgacccgcgaggagcgcaagaagtttgaaaacctgaactcatcctacaacaagtactgggtgccctgcgtctggttctccaacctggcggcacaggcccgacgcgagggccgcatccgcgacaacagcgcccttaagctgctgctcgaggagctgaatgtttttcggggcaaatgtggaatgctctttcactatgactggattagcgtacccctcgtgtacacgcaggtggtgaccatcgcactgtacagctacttcctggcttgcctcattggtcgccagttcctggacccggctcagggttacaaagaccacgacctagacctgtgtgtgcccatcttcaccctcttgcagttcttcttctacgccggctggctcaaggtagctgagcagctcatcaaccccttcggagaggacgatgatgactttgagaccaactttctgatcgatagaaacttccaggtgtccatgctggcagtggacgagatgtatgatgacctggctgtgctggagaaggacttgtactgggatgcagccgaggctcgcgccccatacacagcggctactgtcttccagctgcggcagccttccttccagggctccacctttgacatcacgctggccaaagaagacatgcagttccagcggctggacggcttggatggaccgatgggagaggcgcccggcgacttcctgcagcgcctcctgccggcgggcgcgggcatggtcgcgggaggcccgctgggccggcgcctgtcctttctactccgcaagaacagctgcgtgtcggaggcgtctactggggccagctgctcatgcgcggttgtccccgaaggcgcggccccggagtgcagctgcggggacccgctgctcgaccccggcctgccggagcccgaggccccgccccctgcgggtcccgaaccgcttaccctcatccctgggcctgtcgagcccttcagcatcgtgaccatgcccgggccccggggtccggcgccaccctggctgcccagccctattggcgaggaggaggagaatctggcctgagatcttagagcccagccccctaaggacagggaaccaggtccctgcacggcacccacgcaggtgtcccggtctgcataagcctcgtgtgcctttgtaaagtccacctacacttttgaccagctctcgctgcccgcatgtgtttggcgctgtgctaggggcgggagttcttccagactcttggaccagcccgccctgaccaccagctctacttcccaacccccactgcctgagaggtctctatcagtgtcctgcctgaattctttccttcaagtgaagatgtgactgactacctcctcgagttgtcatgaggatgaaagaatggcattaaagcatttggtatacagtaggtgctcaataaattgctagtgatttttactcaaaaaaaaaaaaaaaa

SEQ ID NO: 2 - protein, 509 aa, Homo sapiens
MTVTYTARVANARFGGFSQLLLLWRGSIYKLLWRELLCFLGFYMALSAAYRFVLTEGQKRYFEKLVIYCDQYASLIPVSFVLGFYVTLVVNRWWSQYLCMPLPDALMCVVAGTVHGRDDRGRLYRRTLMRYAGLSAVLILRSVSTAVFKRFPTIDHVVEAGFMTREERKKFENLNSSYNKYWVPCVWFSNLAAQARREGRIRDNSALKLLLEELNVFRGKCGMLFHYDWISVPLVYTQVVTIALYSYFLACLIGRQFLDPAQGYKDHDLDLCVPIFTLLQFFFYAGWLKVAEQLINPFGEDDDDFETNFLIDRNFQVSMLAVDEMYDDLAVLEKDLYWDAAEARAPYTAATVFQLRQPSFQGSTFDITLAKEDMQFQRLDGLDGPMGEAPGDFLQRLLPAGAGMVAGGPLGRRLSFLLRKNSCVSEASTGASCSCAVVPEGAAPECSCGDPLLDPGLPEPEAPPPAGPEPLTLIPGPVEPFSIVTMPGPRGPAPPWLPSPIGEEEENLA

SEQ ID NO: 3 - DNA, 2089 bp, Mus musculus
  CDS (204)..(1730)
tctctgtcccttgtgtccctgtacggccacaggtgagaagggccaggtctgggagaataaatggccctggactggcaggctttgggcaggactcctggagagagaggggcagagtggaaggctaggaggggcagcacaaaaggaagaaaggcccggacccaggctaaaaccttctccattggccacactggccaggcgcagcgatgaccgtcacctacacagccagagtggcgaatgcccgcttcggtggcttctcgcagctgctgctgctgtggcgcggaagcatctacaagctcctgtggcgagagctgttatgtttcctgggactctacatggcactaagcgccgcctatcgcttcttactggcagaagagcagaagcgctacttcgagaagcttgtcatatactgcgaccagtacgccagcctcatccccgtctctttcgtacttggcttctacgtgactctggtggtgcatcgctggtggaaccagtacctatgcatgcctctgccggacgcactcatgtgcatagtggctggcaccgtgcatgggcgagacgatcgaggccgcctctaccggcgcacgctcatgcgctacgcagggctctccgcggtgctgatccttcgttctgttagcacagcagtcttcaaacggttccccactatagaccacgtggtcgaggctggatttatgacccgagaagagcgcaagaagttcgagaacttgaattcgtcctacaacaaatactgggtgccctgcgtatggttctcgagcctggcagcgcaggcgcggcgagaggggcgcatccgcgacaacagtgccctaaagttgctactagaggagctgaatgtgtttcggagcaaatgtgggatgctgtttcactacgactggattagcatacccctcgtctacacccaggtagtcactatcgcagtgtacagctacttcttggcttgcctcatcggccgtcagttcctagaccctgcacagggctacaaagaccacaccctggacctatgcgtacccatcttcaccttgctgcagttcttcttctacgctggctggcttaaggtagcagagcagcttattaacccttttggggaagacgacgacgactttgagaccaactttcttattgaccgcaacttccaggtgtccatgctagctgtagatgagatgtatgacgacttggccatgctggagaaagatctatactgggatgcagcagaagctcgcgcgccctacaccgcggccaccgctttcctgcttcagcagccctccttccagggctccacctttgatatagcgctggctaaagaggacatgcagttccagcggctggacggcgtggatggcccgctgggagaggttcacggggactttctgcagcgcctcctgccggcgggggcgggttctgtgggtccgctggggcggcgcctgtcgctgttaaggcgtaaaaacagctgcgtgtcagaggcctccactgctgccagctgtggctgtgcaggcgcggcggatggcggaggcgttgagtgcggttgtggagacccattgcttgaccccagcctgcgggagcctgagctggagtctcccgcatgtcctgagccgcctgcccccatcccggggccaactcctgaaccttttaccaccgtgtcgattccgggtcctcgggctcctgcgccaccctggctgcccagccctattggagaggaggaagagagtccggcctgagatcctgggaccaggacccctacggacacctacctaatcctgcactagacatgctggcgaccaccagggtctgcacaagctagctaggccttcagaaagccgacctactcattttgaccagcggaatctcccactggggctggtggttttaggggtccatattctcccagctgttgctgcttccctcacccccaaccccccactctttaagaggttttgccttccaaacaccaccacctgacctgtggcctctgttggtcttctgaccgtaccttgggttcttcccttcgagtggaattgacggcctacctcatgaaattgtgaggatgaaaaaaataaataaaataaaataaacgaaa

SEQ ID NO: 4 - protein, 508 aa, Mus musculus
MTVTYTARVANARFGGFSQLLLLWRGSIYKLLWRELLCFLGLYMALSAAYRFLLAEEQKRYFEKLVIYCDQYASLIPVSFVLGFYVTLVVHRWWNQYLCMPLPDALMCIVAGTVHGRDDRGRLYRRTLMRYAGLSAVLILRSVSTAVFKRFPTIDHVVEAGFMTREERKKFENLNSSYNKYWVPCVWFSSLAAQARREGRIRDNSALKLLLEELNVFRSKCGMLFHYDWISIPLVYTQVVTIAVYSYFLACLIGRQFLDPAQGYKDHTLDLCVPIFTLLQFFFYAGWLKVAEQLINPFGEDDDDFETNFLIDRNFQVSMLAVDEMYDDLAMLEKDLYWDAAEARAPYTAATAFLLQQPSFQGSTFDIALAKEDMQFQRLDGVDGPLGEVHGDFLQRLLPAGAGSVGPLGRRLSLLRRKNSCVSEASTAASCGCAGAADGGGVECGCGDPLLDPSLREPELESPACPEPPAPIPGPTPEPFTTVSIPGPRAPAPPWLPSPIGEEEESPA